(19) United States
(12) Patent Application Publication
(10) Pub. No.: US 2022/0325238 A1
(43) Pub. Date: Oct. 13, 2022

(54) WNT COMPOSITIONS AND METHODS FOR SERUM-FREE SYNTHESIS

(71) Applicant: The Board of Trustees of the Leland Stanford Junior University, Stanford, CA (US)

(72) Inventors: Jill Helms, Stanford, CA (US); Girija Dhamdhere, Stanford, CA (US); Bo Liu, Palo Alto, CA (US); Andrew A. Smith, Seattle, WA (US); Alan W. Gomez, Lake Oswego, OR (US)

(21) Appl. No.: 17/698,636

(22) Filed: Mar. 18, 2022

Related U.S. Application Data
(63) Continuation of application No. 16/067,944, filed on Jul. 3, 2018, now abandoned.
(60) Provisional application No. 62/288,365, filed on Jan. 28, 2016.

(51) Int. Cl. C12N 5/071; A61K 9/127; C07K 1/36; C07K 14/47; C07K 19/00; C12N 15/63

(52) U.S. Cl. CPC C12N 5/0602; A61K 9/127; C07K 1/36; C07K 14/47; C07K 19/00; C12N 15/63; A61K 38/00

(57) ABSTRACT

Provided herein are methods and culture systems for production of a biologically active Wnt polypeptide under a minimal serum condition. Also described herein include methods and culture systems for production of a biologically active Wnt polypeptide in a serum-free condition.

### CROSS-REFERENCE

This application is a Continuation and claims the benefit of U.S. application Ser. No. 16/067,944, filed Jul. 3, 2018, which claims the benefit of PCT Application No. PCT/US2017/015312, filed Jan. 27, 2017, which claims the benefit of U.S. Provisional Application No. 62/288,365, filed Jan. 28, 2016, which applications are incorporated herein by reference in their entirety.

### INCORPORATION BY REFERENCE OF SEQUENCE LISTING PROVIDED AS A TEXT FILE

A Sequence Listing is provided herewith in a text file, S15-464_STAN-1218_Seq_List_ST25, created on Mar. 18, 2022 and having a size of 19,618 bytes. The contents of the text file are incorporated herein by reference in its entirety.

### BACKGROUND OF THE INVENTION

Wnt proteins form a family of highly conserved secreted signaling molecules that bind to cell surface receptors encoded by the Frizzled and low-density lipoprotein receptor related proteins (LRPs). The WNT gene family consists of structurally related genes which encode secreted signaling proteins. These proteins have been implicated in oncogenesis and in several developmental processes, including regulation of cell fate and patterning during embryogenesis. Once bound, the ligands initiate a cascade of intracellular events that eventually lead to the transcription of target genes through the nuclear activity of β-catenin and the DNA binding protein TCF (Clevers H, 2004 Wnt signaling: Ig-norrin the dogma. Curr Biol 14: R436-R437; Nelson & Nusse 2004 Convergence of Wnt, beta-catenin, and cadherin pathways. Science 303: 1483-1487; Gordon & Nusse 2006 Wnt signaling: Multiple pathways, multiple receptors, and multiple transcription factors. J Biol Chern 281: 22429-22433).

Wnts are also involved in a wide variety of cellular decisions associated with the program of osteogenesis. For example, Wnts regulate the expression level of sox9, which influences the commitment of mesenchymal progenitor cells to a skeletogenic fate. Wnts influence the differentiation of cells, into either osteoblasts or chondrocytes. In adult animals, there is abundant evidence that Wnt signaling regulates bone mass. For example, mutations in the human Wnt co-receptor LRPS are associated with several high bone mass syndromes, including osteoporosis type I, and endosteal hyperostosis or autosomal dominant osteosclerosis, as well as a low bone mass disease, osteoporosis-pseudoglioma. Increased production of the Wnt inhibitor DkkI is associated with multiple myeloma, a disease that has increased bone resorption as one of its distinguishing features.

### SUMMARY OF THE INVENTION

Disclosed herein, in certain embodiments, are methods and culture systems of producing a biologically active Wnt polypeptide under a minimal serum condition (e.g., a serum-free condition). Also disclosed herein, in certain embodiments, are compositions that comprise cells engineered to secrete biologically active Wnt polypeptides into a minimal serum culture medium (e.g., serum-free culture medium).

Disclosed herein, in certain embodiments, is an in vitro method of producing a biologically active Wnt polypeptide under a minimal serum condition, which comprises culturing cells from an engineered cell line transfected with an expression vector encoding a Wnt polypeptide under the minimal serum condition, and collecting secreted Wnt polypeptide from the culture medium under the minimal serum condition. In some embodiments, the engineered cell line is a cGMP-compatible cell line. In some embodiments, the cGMP-compatible cell line is a cGMP-compatible mammalian cell line. In some embodiments, the cGMP-compatible mammalian cell line is Chinese Hamster Ovary (CHO) cell line, human embryonic kidney (HEK) cell line, or baby hamster kidney (BHK) cell line. In some embodiments, the cGMP-compatible cell line is a cGMP-compatible insect cell line. In some embodiments, the cGMP-compatible insect cell line is Sf9 cell line, Sf21 cell line, Tn-368 cell line, or High Five (BTI-TN-5B1-4) cell line. In some embodiments, the cells are grown as adherent or suspension culture. In some embodiments, the cells are grown for up to 2, 3, 4, 5, 6, 7, 8, 9, 10, 11, 12, 13, 14, or 15 days prior to collecting the secreted Wnt polypeptide from the culture medium. In some embodiments, the expression vector is a cGMP-compatible vector. In some embodiments, the expression vector is a mammalian vector. In some embodiments, the mammalian vector is OpticVec™ (expression vector), pTarget™ (expression vector), pcDNA™4 TO4 (expression vector), pcDNA™4.0 (expression vector), UCOE® expression vector, or GS System expression Vector™. In some embodiments, the expression vector is an insect cell expression vector. In some embodiments, the insect cell expression vector is pIEx™ or pBiEx™ vectors. In some embodiments, the Wnt polypeptide comprises a heterologous signal sequence. In some embodiments, the Wnt polypeptide comprises a native signal sequence. In some embodiments, the Wnt polypeptide is a Wnt3A polypeptide, Wnt5B polypeptide, or Wnt10B polypeptide. In some embodiments, the Wnt polypeptide is a Wnt3A polypeptide. In some embodiments, the Wnt3A polypeptide is polypeptide that comprises about 90%, 95%, 99%, or more sequence identity to SEQ ID NO: 1. In some embodiments, the Wnt3A polypeptide is polypeptide that comprises about 1 to about 33 amino acid truncations. In some embodiments, the truncation is a C-terminal truncation. In some embodiments, the Wnt3A polypeptide is a polypeptide of SEQ ID NO: 1 with a C-terminal truncation. In some embodiments, the Wnt3A polypeptide is a polypeptide that comprises about 90%, 95%, 99%, or more sequence identity to SEQ ID NO: 2. In some embodiments, the Wnt3A polypeptide is a polypeptide consisting of SEQ ID NO: 2. In some embodiments, the Wnt3A polypeptide is secreted into the culture medium at a concentration of at least about 10 ng/m L. In some embodiments, the minimal serum condition comprises reduced-serum media, protein-free media, chemically defined media, or serum-free media. In some embodiments, the minimal serum condition comprises an animal-component free medium. In some embodiments, the minimal serum condition comprises a culture medium that is substantially free of non-human serum. In some embodiments, the minimal serum condition comprises a culture medium that is substantially free of non-human proteins. In some embodiments, the minimal serum condition comprises a culture medium with less than about 9%, 8%, 7%, 6%, 5%, 4%, 3%, 2%, 1%, or 0.5% serum. In some embodiments, the minimal serum condition comprises a culture medium with 0% serum. In some embodiments, the serum is fetal bovine serum. In some embodiments, the culture medium further comprises serum substitutes. In some embodiments, the serum substitutes comprise CellEss®, ITS (Insulin-Transferrin-Selenium) (e.g., ITS3 or ITS3+), Excyte®, OneShot™ (One Shot fetal bovine serum), or Knockout™ Media. In some embodiments, the culture medium is substantially free of adventitious agents. In some embodiments, the adventitious agents comprise pathogens, transmissible spongiform encephalophathy (TSE) agents, or combinations thereof. In some embodiments, the method further comprises purifying the Wnt polypeptide utilizing an ion-exchange method, a hydrophobic purification method, or an affinity purification method. In some embodiments, the method further comprises formulating the purified Wnt polypeptide with a liposome. In some embodiments, the method further comprises formulating the purified Wnt polypeptide with a pharmaceutically acceptable excipient. A biologically active Wnt polypeptide produced by the in vitro method discussed above.

Disclosed herein, in certain embodiments, is a Wnt culture system which comprises minimal serum culture medium, a biologically active Wnt polypeptide secreted into the minimal serum culture medium, and cells from an engineered cell line transfected with an expression vector encoding the biologically active Wnt polypeptide, wherein the cells are grown in the presence of the minimal serum culture medium. In some embodiments, the engineered cell line is a cGMP-compatible cell line. In some embodiments, the cGMP-compatible cell line is a cGMP-compatible mammalian cell line. In some embodiments, the cGMP-compatible mammalian cell line is Chinese Hamster Ovary (CHO) cell line, human embryonic kidney (HEK) cell line, or baby hamster kidney (BHK) cell line. In some embodiments, the cGMP-compatible cell line is a cGMP-compatible insect cell line. In some embodiments, the cGMP-compatible insect cell line is Sf9 cell line, Sf21 cell line, Tn-368 cell line, or High Five (BTI-TN-5B1-4) cell line. In some embodiments, the expression vector is a cGMP-compatible vector. In some embodiments, the expression vector is a mammalian vector. In some embodiments, the mammalian vector is OpticVec™ (expression vector), pTarget™ (expression vector), pcDNA™4 TO4, pcDNA™4.0, UCOE® expression vector, or GS System expression Vector™. In some embodiments, the expression vector is an insect cell expression vector. In some embodiments, the insect cell expression vector is pIEx™ or pBiEx™ vectors. In some embodiments, the Wnt polypeptide comprises a heterologous signal sequence. In some embodiments, the Wnt polypeptide comprises a native signal sequence. In some embodiments, the Wnt polypeptide is a Wnt3A polypeptide, Wnt5B polypeptide, or Wnt10B polypeptide. In some embodiments, the Wnt polypeptide is a Wnt3A polypeptide. In some embodiments, the Wnt3A polypeptide is polypeptide that comprises about 90%, 95%, 99%, or more sequence identity to SEQ ID NO: 1. In some embodiments, the Wnt3A polypeptide is polypeptide that comprises about 1 to about 33 amino acid truncations. In some embodiments, the truncation is a C-terminal truncation. In some embodiments, the Wnt3A polypeptide is a polypeptide of SEQ ID NO: 1 with a C-terminal truncation. In some embodiments, the Wnt3A polypeptide is a polypeptide that comprises about 90%, 95%, 99%, or more sequence identity to SEQ ID NO: 2. In some embodiments, the Wnt3A polypeptide is a polypeptide consisting of SEQ ID NO: 2. In some embodiments, the concentration of the secreted biologically active Wnt3A polypeptide is at least about 10 ng/mL in the culture medium. In some embodiments, the culture medium is about 2, 3, 4, 5, 6, 7, 8, 9, 10, 11, 12, 13, 14, or 15 days old. In some embodiments, the culture medium is reduced-serum media, protein-free media, chemically defined media, or serum-free media. In some embodiments, the culture medium is an animal-component free medium. In some embodiments, the culture medium is substantially free of non-human serum. In some embodiments, the culture medium is substantially free of non-human proteins. In some embodiments, the culture medium comprises less than about 9%, 8%, 7%, 6%, 5%, 4%, 3%, 2%, 1%, or 0.5% serum. In some embodiments, the culture medium comprises 0% serum. In some embodiments, the serum is fetal bovine serum. In some embodiments, the culture medium further comprises serum substitutes. In some embodiments, the serum substitutes comprise CellEss®, ITS (Insulin-Transferrin-Selenium), Excyte®, OneShot™ (One Shot fetal bovine serum), or Knockout™ Media. In some embodiments, the culture medium is substantially free of adventitious agents. In some embodiments, the adventitious agents comprise pathogens, transmissible spongiform encephalophathy (TSE) agents, or combinations thereof.

Disclosed herein, in certain embodiments, is a culture medium which comprises minimal serum culture medium, a biologically active Wnt polypeptide secreted into the minimal serum culture medium, and cells from an engineered cell line transfected with an expression vector encoding the biologically active Wnt polypeptide, wherein the cells are grown in the presence of the minimal serum culture medium. In some embodiments, the engineered cell line is a cGMP-compatible cell line. In some embodiments, the cGMP-compatible cell line is a cGMP-compatible mammalian cell line. In some embodiments, the cGMP-compatible mammalian cell line is Chinese Hamster Ovary (CHO) cell line, human embryonic kidney (HEK) cell line, or baby hamster kidney (BHK) cell line. In some embodiments, the cGMP-compatible cell line is a cGMP-compatible insect cell line. In some embodiments, the cGMP-compatible insect cell line is Sf9 cell line, Sf21 cell line, Tn-368 cell line, or High Five (BTI-TN-5B1-4) cell line. In some embodiments, the expression vector is a cGMP-compatible vector. In some embodiments, the expression vector is a mammalian vector. In some embodiments, the mammalian vector is OpticVec™ (expression vector), pTarget™ (expression vector), pcDNA™4TO4, pcDNA™4.0, UCOE® expression vector, or GS System expression Vector™. In some embodiments, the expression vector is an insect cell expression vector. In some embodiments, the insect cell expression vector is pIEx™ or pBiEx™ vectors. In some embodiments, the Wnt polypeptide comprises a heterologous signal sequence. In some embodiments, the Wnt polypeptide comprises a native signal sequence. In some embodiments, the Wnt polypeptide is a Wnt3A polypeptide, Wnt5B polypeptide, or Wnt10B polypeptide. In some embodiments, the Wnt polypeptide is a Wnt3A polypeptide. In some embodiments, the Wnt3A polypeptide is polypeptide that comprises about 90%, 95%, 99%, or more sequence identity to SEQ ID NO: 1. In some embodiments, the Wnt3A polypeptide is polypeptide that comprises about 1 to about 33 amino acid truncations. In some embodiments, the truncation is a C-terminal truncation. In some embodiments, the Wnt3A polypeptide is a polypeptide of SEQ ID NO: 1 with a C-terminal truncation. In some embodiments, the Wnt3A polypeptide is a polypeptide that comprises about 90%, 95%, 99%, or more sequence identity to SEQ ID NO: 2. In some embodiments, the Wnt3A polypeptide is a polypeptide consisting of SEQ ID NO: 2. In some embodiments, the concentration of the secreted biologically active Wnt3A polypeptide is at least about 10 ng/mL in the culture medium. In some embodiments, the culture medium is about 2, 3, 4, 5, 6, 7, 8, 9, 10, 11, 12, 13, 14, or 15 days old. In some embodiments, the culture medium is reduced-serum media, protein-free media, chemically defined media, or serum-free media. In some embodiments, the culture medium is an animal-component free medium. In some embodiments, the culture medium is substantially free of non-human serum. In some embodiments, the culture medium is substantially free of non-human proteins. In some embodiments, the culture medium comprises less than about 9%, 8%, 7%, 6%, 5%, 4%, 3%, 2%, 1%, or 0.5% serum. In some embodiments, the culture medium comprises 0% serum. In some embodiments, the serum is fetal bovine serum. In some embodiments, the culture medium further comprises serum substitutes. In some embodiments, the serum substitutes comprise CellEss®, ITS (Insulin-Transferrin-Selenium), Excyte®, OneShot™ (One Shot fetal bovine serum), or Knockout™ Media. In some embodiments, the culture medium is substantially free of adventitious agents. In some embodiments, the adventitious agents comprise pathogens, transmissible spongiform encephalophathy (TSE) agents, or combinations thereof.

Disclosed herein, in certain embodiments, is a method of preparing a liposomal Wnt polypeptide, comprising: (a) incubating an isolated Wnt polypeptide with a plurality of chaperones to generate a Wnt polypeptide-chaperone complex; (b) separating the Wnt polypeptide-chaperone complex from non-complexed chaperones; and (c) contacting the Wnt polypeptide-chaperone complex with an aqueous solution of liposomes to generate the liposomal Wnt polypeptide. In some embodiments, the plurality of chaperones comprise Frizzled-8. In some embodiments, each chaperone from the plurality of chaperones comprises a Frizzled-8 fusion protein. In some embodiments, the Frizzled-8 fusion protein comprises a truncated Frizzled-8 protein. In some embodiments, the truncated Frizzled-8 protein comprises a cysteine-rich region (CRD) of Frizzled-8. In some embodiments, the truncated Frizzled-8 protein comprises the region spanning amino acid residue 25 to amino acid residue 172 of SEQ ID NO: 4. In some embodiments, the Frizzled-8 fusion protein further comprises an IgG Fc portion. In some embodiments, the Frizzled-8 fusion protein comprises at least 80%, 85%, 90%, 95%, 96%, 97%, 98%, or 99% sequence identity to SEQ ID NO: 5. In some embodiments, the isolated Wnt polypeptide and the plurality of chaperones are incubated for at least 10 minutes, at least 30 minutes, at least 1 hour, at least 1.5 hour, at least 2 hours, at least 3 hours, at least 4 hours, at least 5 hours, at least 6 hours, at least 10 hours, at least 12 hours, at least 18 hours, or more. In some embodiments, the isolated Wnt polypeptide and the plurality of chaperones are incubated at a temperature of between about 1° C. and about 30° C. In some embodiments, the isolated Wnt polypeptide and the plurality of chaperones are incubated at a temperature of between about 1° C. and about 10° C., between about 1° C. and about 8° C., or between about 1° C. and about 4° C. In some embodiments, the isolated Wnt polypeptide and the plurality of chaperones are incubated at a temperature of between about 10° C. and about 30° C., between about 15° C. and about 30° C., between about 20° C. and about 30° C., between about 23° C. and about 30° C., or between about 25° C. and about 30° C. In some embodiments, the isolated Wnt polypeptide and the plurality of chaperones are incubated at a temperature of at least 1° C., 2° C., 4° C., 8° C., 10° C., 20° C., 23° C., 25° C. or 30° C. In some embodiments, each of the plurality of chaperones is further immobilized on a bead. In some embodiments, each of the plurality of chaperones is further immobilized indirectly on a bead, wherein each chaperone is bound to a polypeptide that recognizes the Fc portion of an antibody, and wherein the polypeptide is immobilized to the bead. In some embodiments, the polypeptide is Protein A. In some embodiments, the isolated Wnt polypeptide and the plurality of chaperones are incubated at a ratio of about 1:0.5, 1:1, 1:1.5, 1:2, 1:2.5, 1:3, 1:4, or about 1:5 Wnt polypeptide:chaperone. In some embodiments, the Wnt polypeptide and the plurality of chaperones are incubated at a ratio of about 1:2.5 Wnt polypeptide:chaperone. In some embodiments, the separating of step b) comprises eluting the isolated Wnt polypeptide-chaperone complex with a buffer comprising a pH of about 3.0. In some embodiments, a phospholipid comprising the liposome has a tail carbon length of between about 12 carbons and about 14 carbons. In some embodiments, the liposomes have a net charge of 0 at a pH of between about 6.5 and about 8.0, about 7.0 and about 7.8, or about 7.2 and about 7.6. In some embodiments, the phospholipid is 1,2-dimyristoyl-sn-glycero-3-phosphocholine (DMPC). In some embodiments, the liposome further comprises cholesterol. In some embodiments, the concentration of DMPC and cholesterol is defined by a ratio of between about 70:30 and about 100:0. In some embodiments, the incubating of step a) further comprises harvesting the isolated Wnt polypeptide from a Wnt culture system described herein. In some embodiments, the isolated Wnt polypeptide is an isolated Wnt5B polypeptide or an isolated Wnt10B polypeptide. In some embodiments, the isolated Wnt polypeptide is an isolated Wnt3A polypeptide.

Disclosed herein, in certain embodiments, is a method of purifying a Wnt polypeptide, comprising: (a) incubating a liposomal Wnt polypeptide with a plurality of chaperones to form a liposomal Wnt polypeptide-chaperone complex; (b) separating the liposomal Wnt polypeptide-chaperone complex from non-complexed chaperones; and (c) eluting the liposomal Wnt polypeptide from the liposomal Wnt polypeptide-chaperone complex to generate a purified liposomal Wnt polypeptide. In some embodiments, the plurality of chaperones comprise Frizzled-8. In some embodiments, each chaperone from the plurality of chaperones comprises a Frizzled-8 fusion protein. In some embodiments, the Frizzled-8 fusion protein comprises a truncated Frizzled-8 protein. In some embodiments, the truncated Frizzled-8 protein comprises a cysteine-rich region (CRD) of Frizzled-8. In some embodiments, the truncated Frizzled-8 protein comprises the region spanning amino acid residue 25 to amino acid residue 172 of SEQ ID NO: 4. In some embodiments, the Frizzled-8 fusion protein further comprises an IgG Fc portion. In some embodiments, the Frizzled-8 fusion protein comprises at least 80%, 85%, 90%, 95%, 96%, 97%, 98%, or 99% sequence identity to SEQ ID NO: 5. In some embodiments, the plurality of chaperones comprise low-density lipoprotein receptor-related protein 6 (Lrp6). In some embodiments, the liposomal Wnt polypeptide and the plurality of chaperones are incubated for at least 10 minutes, at least 30 minutes, at least 1 hour, at least 1.5 hour, at least 2 hours, at least 3 hours, at least 4 hours, at least 5 hours, at least 6 hours, at least 10 hours, at least 12 hours, at least 18 hours, or more. In some embodiments, the liposomal Wnt polypeptide and the plurality of chaperones are incubated at a temperature of between about 1° C. and about 30° C. In some embodiments, the liposomal Wnt polypeptide and the plurality of chaperones are incubated at a temperature of between about 1° C. and about 10° C., between about 1° C. and about 8° C., or between about 1° C. and about 4° C. In some embodiments, the liposomal Wnt polypeptide and the plurality of chaperones are incubated at a temperature of between about 10° C. and about 30° C., between about 15° C. and about 30° C., between about 20° C. and about 30° C., between about 23° C. and about 30° C., or between about 25° C. and about 30° C. In some embodiments, the liposomal Wnt polypeptide and the plurality of chaperones are incubated at a temperature of at least 1° C., 2° C., 4° C., 8° C., 10° C., 20° C., 23° C., 25° C., or 30° C. In some embodiments, the Frizzled-8 fusion protein is further immobilized on a bead. In some embodiments, the Frizzled-8 fusion protein is further immobilized indirectly on a bead, wherein the Frizzled-8 fusion protein is bound to a polypeptide that recognizes the Fc portion, and wherein the polypeptide is immobilized on the bead. In some embodiments, the polypeptide is Protein A. In some embodiments, the liposomal Wnt polypeptide and the plurality of chaperones are incubated at a ratio of about 1:0.5, 1:1, 1:1.5, 1:2, 1:2.5, 1:3, 1:4, or about 1:5 Wnt polypeptide:chaperone. In some embodiments, the separating of step b) comprises eluting the liposomal Wnt polypeptide-chaperone complex with a buffer, wherein the buffer optionally comprises a pH of about 3.0. In some embodiments, a phospholipid comprising the liposome has a tail carbon length of between about 12 carbons and about 14 carbons. In some embodiments, the liposomes have a net charge of 0 at a pH of between about 6.5 and about 8.0, about 7.0 and about 7.8, or about 7.2 and about 7.6. In some embodiments, the phospholipid is 1,2-dimyristoyl-sn-glycero-3-phosphocholine (DMPC). In some embodiments, the liposome further comprises cholesterol. In some embodiments, the concentration of DMPC and cholesterol is defined by a ratio of between about 70:30 and about 100:0. In some embodiments, the incubating in step a) further comprises contacting an isolated Wnt polypeptide obtained from a Wnt culture system described herein with an aqueous solution of liposomes to generate the liposomal Wnt polypeptide. In some embodiments, the isolated Wnt polypeptide is an isolated Wnt5B polypeptide or an isolated Wnt10B polypeptide. In some embodiments, the isolated Wnt polypeptide is an isolated Wnt3A polypeptide.

Further aspects and embodiment will be apparent from the rest of the disclosure, and are included within the present invention.

### BRIEF DESCRIPTION OF THE DRAWINGS

FIG. 1 illustrates Wnt3A activity in the presence of serum substitute Excyte® and decreasing serum concentrations. Wnt polypeptide is from an expression vector encoding the protein sequence set forth in SEQ ID NO:1. WNT3a activity in conditioned media from cells adapted to 5% serum+Excyte® (blue dashed bar), 3% serum+Excyte® (red dashed bar) and 2% serum+Excyte® (purple dashed bar) was analyzed using dual light reporter assay. This activity was compared to activity of conditioned media from cells adapted to 5% serum (blue solid bar), 3% serum (red solid bar) and 2% serum (purple solid bar) without Excyte® supplement. The condition media from cells grown in 10% serum (orange bar) was used as a positive control. As compared to 10% FBS the activity of conditioned media from cells adapted to 2% serum and 2% serum+Excyte® was reduced to 6.4%. Decreasing serum concentrations resulted in reduced Wnt3A activity in the conditioned media. Addition of Excyte® did not have an effect on Wnt3A activity in conditioned media.

FIG. 2 shows Wnt3A activity in the presence of serum substitute CellEss® and decreasing serum concentrations. The Wnt3A polypeptide is from an expression vector encoding the protein sequence set forth in SEQ ID NO:1. Wnt3A activity in conditioned media from cells adapted to 7.5% and 5% serum supplemented with Excyte® was analyzed using a dual light reporter assay. This activity was compared to Wnt3A activity in condition media from cells grown in 10% serum. Presence of CellEss® in the culture media was not able to restore Wnt3A activity in the conditioned media.

FIG. 3 shows Wnt3A activity from an expression vector encoding the protein sequence set forth in SEQ ID NO:1. Cells were first adapted to charcoal stripped one shot FBS (OS FBS). No detectable activity was measured in conditioned media from cells adapted to OSFBS. Following adaptation to OSFBS, OSFBS was supplemented with either ITS3 or lipid mix 1. WNT3A activity in conditioned media was tested using the LSL dual light reporter assay. Conditioned media from cells adapted to OSFBS+ITS (Insulin-Transferrin-Selenium) sample demonstrated ˜10% of activity when compared to the positive control (10% FBS). Conditioned media from cells adapted to OSFBS+lipid mix sample demonstrated 26% of activity when compared to the positive control, 10% FBS.

FIG. 4 shows Wnt3A polypeptide secretion by cells under different serum conditions. Cells were grown in 10%, 1% and 0% serum containing conditions. Cells were induced and conditioned media was collected over a period of 5 days (d2, d3 and d5).

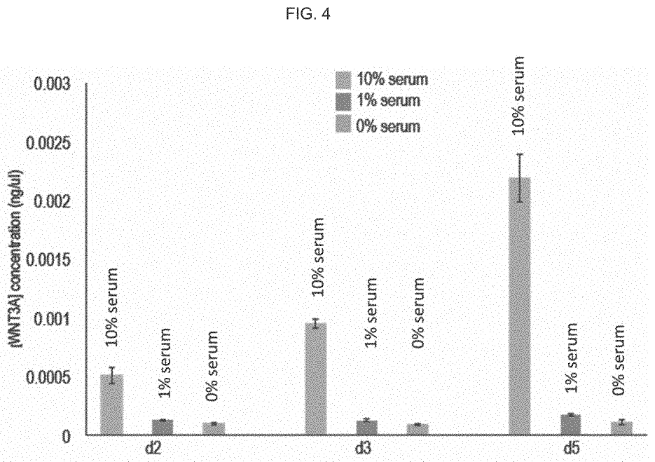

FIGS. 5A-5B illustrate activity of Wnt3A secreted by CHO cells under serum free conditions. FIG. 5A illustrates a LSL reporter assay. FIG. 5B illustrates a Western blot analysis to detect the presence of Wnt3A.

FIG. 6 illustrates Wnt3A activity from a stably transfected CHO-S cell line grown under serum free conditions.

FIG. 7 illustrates a schematic for purification of a Wnt polypeptide utilizing a chaperone described herein.

FIG. 8 illustrates a schematic showing a pre-complexation of a Frizzled-8 fusion protein with Protein A immobilized beads.

FIG. 9 illustrates a western blot showing complexation of Frizzled-8-Fc to Protein A at two different ratios.

FIG. 10 illustrates a western blot showing Wnt3A purified using the Frizzled-8 fusion protein-Protein A strategy.

FIG. 11A-FIG. 11C show Fz8 and liposomes compete for binding to Wnt3A. FIG. 11A shows liposomes and Wnt3A were incubated for 6 h at room temperature followed by ultracentrifugation to create L-Wnt3A. Then, this preformed L-Wnt3A was incubated with Fz8 for 6 h at room temperature and then ultracentrifuged to separate liposome-associated proteins from unassociated proteins. Immunoblotting shows that almost all the Fz8 (98.6%) in the supernatant, whereas Wnt3A was only detected in the liposomal pellet. FIG. 11B shows that Fz8 and Wnt3A were pre-incubated for 24 h at 4° C., and then this Fz8—Wnt3A solution was incubated with liposomes for 6 h at room temperature followed by ultracentrifugation. Under these conditions, Fz8 is observed to remain in the supernatant (99.6%), but the majority of Wnt3A (93.0%) is observed to co-localize with Fz8 in the supernatant. FIG. 11C shows Wnt3A, Fz8, and liposomes were incubated together for 6 h at room temperature followed by ultracentrifugation. Fz8 is observed to remain in the supernatant (91.8%), but Wnt3A partitions 62.1% into the liposomal pellet and 37.9% into the supernatant. Data are mean±SEM from, or are representative of, at least three independent replicates.

FIG. 12A-FIG. 12C shows incubation of human Wnt3A, mouse Fz8, and liposomes under three different conditions. FIG. 12A shows liposomes and Wnt3A were incubated for 12 h at room temperature followed by ultracentrifugation to create L-Wnt3A. Then, this preformed L-Wnt3A was incubated with Fz8 for 6 h at room temperature and then ultracentrifuged to separate liposome-associated proteins from unassociated proteins. Immunoblotting showed that about 94.5% of Fz8 was in the supernatant, whereas about 88.7% of Wnt3A was detected in the liposomal pellet. FIG. 12B showed that Fz8 and Wnt3A were pre-incubated for 24 h at 4° C., and then this Fz8—Wnt3A solution was incubated with liposomes for 12 h at room temperature followed by ultracentrifugation. Under these conditions, the majority of Fz8 is observed to remain in the supernatant (72.8%), but the majority of Wnt3A (65.7%) is observed to co-localize with Fz8 in the supernatant. FIG. 12C showed that Wnt3A, Fz8, and liposomes are incubated for 12 h at room temperature followed by ultracentrifugation. Fz8 is observed to remain in the supernatant (94.0%), but Wnt3A is observed to partition 29.9% into the liposomal pellet and 70.1% into the supernatant. Data are mean±SEM from, or are representative of, at least three independent replicates.

FIG. 13A-FIG. 13C shows a binding complex of Wnt3A, LRP6, and liposomes. FIG. 13A shows liposomes and Wnt3A were incubated for 6 h at room temperature followed by ultracentrifugation to create L-Wnt3A. Then, this preformed L-Wnt3A was incubated with LRP6 for 6 h at room temperature and then ultracentrifuged to separate liposome-associated proteins from unassociated proteins. Immunoblotting shows that LRP6 partitions 61.7% in the pellet and 38.3% in the supernatant, whereas Wnt3A is detected in the liposomal pellet. FIG. 13B shows LRP6 and Wnt3A were pre-incubated for 24 h at 4° C., and then this LRP6—Wnt3A solution was incubated with liposomes for 6 h at room temperature followed by ultracentrifugation. Under these conditions, almost all LRP6 (96.2%) is observed to remain in the supernatant, and Wnt3A is observed to partition 65.9% into the pellet and 34.1% into the supernatant. FIG. 13C shows Wnt3A, LRP6, and liposomes incubated for 6 h at room temperature followed by ultracentrifugation. LRP6 is observed to remain mostly in the supernatant (88.9%), and Wnt3A is observed to partition 79.7% into the liposomal pellet and 20.3% into the supernatant. Data are mean±SEM from, or are representative of, at least three independent replicates.

FIG. 14A-FIG. 14C show incubation of human Wnt3A, mouse LRP6, and liposomes under three different conditions. FIG. 14A shows liposomes and Wnt3A were incubated for 6 h at room temperature followed by ultracentrifugation to create L-Wnt3A. Then, this preformed L-Wnt3A was incubated with LRP6 for 12 h at room temperature and then ultracentrifuged to separate liposome-associated proteins from unassociated proteins. Immunoblotting shows that LRP6 partitions 48.2% in the pellet and 51.8% in the supernatant, whereas Wnt3A is only detected in the liposomal pellet. FIG. 14B shows LRP6 and Wnt3A were pre-incubated for 24 h at 4° C., and then this LRP6—Wnt3A solution was incubated with liposomes for 12 h at room temperature followed by ultracentrifugation. Under these conditions, almost all LRP6 (91.5%) is observed to remain in the supernatant, and Wnt3A is observed to partition 61.5% into the pellet and 38.5% into the supernatant. FIG. 14C shows Wnt3A, LRP6, and liposomes incubation for 12 h at room temperature followed by ultracentrifugation. LRP6 is observed to remains mostly in the supernatant (90.8%), and Wnt3A is observed to partition 70.8% into the liposomal pellet and 29.2% into the supernatant. Data are mean±SEM from, or are representative of, at least three independent replicates.

### DETAILED DESCRIPTION OF THE INVENTION

Wnt polypeptides comprise a family of signaling molecules that orchestrates cellular developmental and biological processes. In some instances, Wnt polypeptides modulate stem cell self-renewal, apoptosis, and cell motility. In other instances, Wnt polypeptides contribute to development, such as for example, tissue homeostasis. The Wnt polypeptide is a highly hydrophobic protein and under some instances (e.g., certain media conditions) has reduced or loses biological function. In some cases, formulation of a Wnt polypeptide with an exogenous agent (e.g., a liposome) allows the Wnt polypeptide to maintain biological function. For example, it has been shown that combining a Wnt polypeptide with a lipid vesicle (e.g., a liposome) produce a Wnt formulation (Morrell N T, Leucht P, Zhao L, Kim J-B, ten Berge D, et al. (2008) Liposomal Packaging Generates Wnt Protein with In Vivo Biological Activity. PLoS ONE 3(8): e2930; and Zhao et al., Controlling the in vivo activity of Wnt liposomes, Methods Enzyrnol 465: 331-47 (2009)) with biological activity (Minear et al., Wnt proteins promote bone regeneration. Sci. Transl. Med. 2, 29ra30 (2010); and Popelut et al., The acceleration of implant osseointegration by liposomal Wnt3A, Biomaterials 31 9173e9181 (2010); U.S. Pat. Nos. 7,335,643 and 7,153,832).

In some instances, Wnt polypeptides are secreted from culture cells in the presence of serum. Serum contains a variety of lipid components, which in some cases stabilize the highly hydrophobic Wnt polypeptide in vitro. The hydrophobicity is based on the presence of glycosylation and palmitoylation, modifications which in some cases are required for Wnt activity. For safety reasons, however, regulatory bodies including the FDA and EMA generally require the removal of all animal products from drugs intended for use in humans. Additionally, fetal bovine serum used in the manufacture of FDA-regulated medical products is prohibited if appropriate procedures have not been followed to prevent contamination with viruses and other pathogens.

Disclosed herein are methods and culture systems of producing Wnt polypeptides under minimal serum condition (e.g., serum-free condition). In some embodiments, disclosed herein is an in vitro method of producing a biologically active Wnt polypeptide under a minimal serum condition, which comprises culturing cells from an engineered cell line transfected with an expression vector encoding a Wnt polypeptide under the minimal serum condition; and collecting secreted Wnt polypeptide from the culture medium under the minimal serum condition. In some instances, also described herein include a culture medium that comprises minimal serum culture medium; a biologically active Wnt polypeptide secreted into the minimal serum culture medium; and cells from an engineered cell line transfected with an expression vector encoding the biologically active Wnt polypeptide, wherein the cells are grown in the presence of the minimal serum culture medium. In additional instances, described herein include methods of preparing liposomal Wnt polypeptides and methods of purifying a Wnt polypeptide obtained from a minimal serum condition with a use of an exogenous chaperone.

In some embodiments, the minimal serum condition is a serum-free condition. In some instances, the present invention is based on the development of a serum-free process for the secretion of biologically active Wnt polypeptide (e.g., human Wnt3A). In some embodiments, disclosed herein is an in vitro method of producing a biologically active Wnt polypeptide under a serum-free condition, which comprises culturing cells from an engineered cell line transfected with an expression vector encoding a Wnt polypeptide under the serum-free condition; and collecting secreted Wnt polypeptide from the culture medium under the serum-free condition. In some instances, also described herein include a culture medium that comprises serum-free culture medium; a biologically active Wnt polypeptide secreted into the serum-free culture medium; and cells from an engineered cell line transfected with an expression vector encoding the biologically active Wnt polypeptide, wherein the cells are grown in the presence of the serum-free culture medium.

In some embodiments, the ability to produce the Wnt polypeptides in serum-free medium has a significant benefit for clinical use. Correspondingly, methods and compositions are provided for the serum-free secretion of human WNT3a and for compositions obtained therefrom.

### Certain Terminology

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

Unless defined otherwise, technical and scientific terms used herein have the same meaning as commonly understood by one of ordinary skill in the art to which this invention belongs. Singleton et al, Dictionary of Microbiology and Molecular Biology 2nd ed., J. Wiley & Sons (New York, N.Y. 1994), provides one skilled in the art with a general guide to many of the terms used in the present application.

All publications mentioned herein are expressly incorporated herein by reference to disclose and describe the methods and/or materials in connection with which the publications are cited.

The methods of the invention, as well as tests to determine their efficacy in a particular patient or application, can be carried out in accordance with the teachings herein using procedures standard in the art. Thus, the practice of the present invention may employ conventional techniques of molecular biology (including recombinant techniques), microbiology, cell biology, biochemistry and immunology within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction” (Mullis et al., eds., 1994); and “Current Protocols in Immunology” (J. E. Coligan et al., eds., 1991); as well as updated or revised editions of all of the foregoing.

As used herein, compounds which are “commercially available” may be obtained from commercial sources including but not limited to Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.), Novabiochem and Argonaut Technology.

Compounds can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

As used herein, minimal serum condition includes serum conditions with reduced serum presence, for example, about 9%, 8%, 7%, 6%, 5%, 4%, 3%, 2%, 1.5%, 1%, 0.5%, 0.25%, 0.2%, 0.1%, 0.05% serum, or less. In some instances, the minimal serum condition comprises from 9% to 0%, from 5% to 0.05%, from 5% to 0.1%, from 5% to 0.25%, from 4% to 0.05%, from 4% to 0.1%, from 4% to 0.2%, from 3% to 0.05%, from 3% to 0.1%, from 3% to 0.2%, from 3% to 0.25%, from 2% to 0.05%, from 2% to 0.01%, from 2% to 0.25%, or from 2% to 0.5% serum. In some instances, the minimal serum condition comprises reduced-serum media, protein-free media, chemically defined media, or serum-free media. In some cases, reduced-serum media comprises about 1% to about 5% serum (e.g., fetal bovine serum). In some cases, protein-free media does not contain any proteins or components of animal origin, but sometimes contain peptides and/or polypeptides obtained from plant hydrolysates. In some cases, chemically defined media comprises recombinant proteins and/or hormones (e.g., recombinant albumin and insulin, and chemically defined lipids) and does not contain fetal bovine serum, bovine serum albumin or human serum albumin. In some cases, a chemically defined media is a protein-free, chemically defined media, which comprises low molecular weight constituents and sometimes also contain synthetic peptides and/or hormones. In some cases, a chemically defined media is a peptide-free, protein-free chemically defined media. In some cases, serum-free media (or defined media) comprises undefined animal-derived products such as serum albumin, hydrolysates, growth factors, hormones, carrier proteins, and attachment factors. In some embodiments, the minimal serum condition used herein refers to a media condition comprising less than 9%, 8%, 7%, 6%, 5%, 4%, 3%, 2%, 1.5%, 1%, 0.5%, 0.25%, 0.2%, 0.1%, or 0.05% serum. In some embodiments, the minimal serum condition used herein refers to a media condition comprising from 9% to 0%, from 5% to 0.05%, from 5% to 0.1%, from 5% to 0.25%, from 4% to 0.05%, from 4% to 0.1%, from 4% to 0.2%, from 3% to 0.05%, from 3% to 0.1%, from 3% to 0.2%, from 3% to 0.25%, from 2% to 0.05%, from 2% to 0.01%, from 2% to 0.25%, or from 2% to 0.5% serum. In some embodiments, the minimal serum condition used herein refers to a reduced-serum media condition. In some embodiments, the minimal serum condition used herein refers to protein-free media condition. In some embodiments, the minimal serum condition used herein refers to a chemically defined media condition. In some embodiments, the minimal serum condition as used herein refers to a serum-free media condition.

### Wnt Polypeptide

Wnt polypeptides or proteins form a family of highly conserved secreted signaling molecules that regulate cell-to-cell interactions during embryogenesis. In some embodiments, Wnt polypeptides include Wnt1, Wnt2, Wnt2b (or Wnt13), Wnt3, Wnt3A, Wnt4, Wnt5A, Wnt5b, Wnt6, Wnt7a, Wnt7b, Wnt8a, Wnt8b, Wnt9a (Wnt14, or Wnt14b), Wnt9b (Wnt14b, or Wnt15), Wnt10A, Wnt10B (or Wnt12), Wnt11, Wnt-16a, and Wnt-16b polypeptide. In some embodiments, a Wnt polypeptide is selected from Wnt3A polypeptide, Wnt5A polypeptide, and Wnt10B polypeptide. In some embodiments, the Wnt polypeptide is Wnt3A polypeptide. In some embodiments, the Wnt polypeptide is Wnt5A polypeptide. In some embodiments, the Wnt polypeptide is Wnt10B polypeptide. The terms “Wnts” or “Wnt gene product” or “Wnt polypeptide” when used herein encompass native sequence Wnt polypeptides, Wnt polypeptide variants, Wnt polypeptide fragments and chimeric Wnt polypeptides.

A “native sequence” polypeptide is one that has the same amino acid sequence as a Wnt polypeptide derived from nature. Such native sequence polypeptides can be isolated from cells producing endogenous Wnt protein or can be produced by recombinant or synthetic means. Thus, a native sequence polypeptide can have the amino acid sequence of, e.g. naturally occurring human polypeptide, murine polypeptide, or polypeptide from any other mammalian species, or from non-mammalian species, e.g. Drosophila, C. elegans, and the like.

The term “native sequence Wnt polypeptide” includes, without limitation, Wnt1, Wnt2, Wnt2b (or Wnt13), Wnt3, Wnt3A, Wnt4, Wnt5A, Wnt5b, Wnt6, Wnt7a, Wnt7b, Wnt8a, Wnt8b, Wnt9a (Wnt14, or Wnt14b), Wnt9b (Wnt14b, or Wnt15), Wnt10A, Wnt10B (or Wnt12), Wnt11, Wnt-16a, and Wnt-16b polypeptide. In some instances, the term “native sequence Wnt polypeptide” includes human Wnt polypeptides. In some cases, the human Wnt polypeptides include human Wnt1, Wnt2, Wnt2b (or Wnt13), Wnt3, Wnt3A, Wnt4, Wnt5A, Wnt5b, Wnt6, Wnt7a, Wnt7b, Wnt8a, Wnt8b, Wnt9a (Wnt14, or Wnt14b), Wnt9b (Wnt14b, or Wnt15), Wnt10A, Wnt10B (or Wnt12), Wnt11, Wnt-16a, and Wnt-16b polypeptide. In some cases, the human Wnt polypeptide is human Wnt3A polypeptides. In some cases, the human Wnt polypeptide is human Wnt5A. In additional cases, the human Wnt polypeptide is human Wnt10B.

In some instances, Wnt1 is referred by the Genbank references NP005421.1 and AAH74799.1. Wnt2 is referred by the Genbank references NP003382.1 and AAH78170.1 In general, Wnt2 is expressed in the brain, thalamus, in both fetal and adult lungs, or in the placenta. Wnt2B has two isoforms and their Genbank reference Nos. are NP004176.2 and NP078613.1, respectively. In some cases, isoform 1 is expressed in adult heart, brain, placenta, lung, prostate, testis, ovary, small intestine and/or colon. In the adult brain, it is mainly found in the caudate nucleus, subthalamic nucleus and thalamus. In some instances, it is also detected in fetal brain, lung and kidney. In some cases, isoform 2 is expressed in fetal brain, fetal lung, fetal kidney, caudate nucleus, testis, and/or cancer cell lines.

Wnt3 and Wnt3A play distinct roles in cell-cell signaling during morphogenesis of the developing neural tube. Wnt3 has the Genbank reference AB060284.1 (see also GenBank Nos. BAB61052.1 and AAI03924.1). Wnt3A has the Genbank accession BC103922 and the accession number BC103921. In some instances, the term “native sequence Wnt protein” or “native sequence Wnt polypeptide” includes the Wnt3A native polypeptides (e.g., polypeptides of accession numbers BC103921 and BC103922) with or without the initiating N-terminal methionine (Met), and with or without the native signal sequence. In some cases, the terms include the 352 amino acids native human Wnt3A polypeptide of SEQ ID NO:2, without or without its N-terminal methionine (Met), and with or without the native signal sequence.

In some embodiments, Wnt4 has the Genbank references NP1 10388.2 and BAC23080.1. Wnt5A has the Genbank references NP003383.1, and NP003383.2. Wnt5b has the Genbank references BAB62039.1 and AAG38659. Wnt6 has the Genbank references NP006513.1 and BAB55603.1. Wnt7a has the Genbank references NP004616.2 and BAA82509.1. In some instances, it is expressed in the placenta, kidney, testis, uterus, fetal lung, fetal brain, or adult brain. Wnt7b has the Genbank references NP478679.1 and BAB68399.1. In some cases, it is expressed in fetal brain, lung and/or kidney, or in adult brain, lung and/or prostate. Wnt8A has at least two alternative transcripts, Genbank references NP114139.1 and NP490645.1. Wnt8B is expressed in the forebrain. It has the Genbank reference NP003384.1. Wnt10A has the Genbank references AAG45153 and NP079492.2. Wnt10B is detected in most adult tissues, with highest levels in the heart and skeletal muscles. It has the Genbank reference NP003385.2. In some cases, Wnt11 is expressed in fetal lung, kidney, adult heart, liver, skeletal muscle, and pancreas. It has the Genbank reference NP004617.2. Wnt14 has the Genbank reference NP003386.1. Wnt15 is expressed in fetal kidney or adult kidney, or expressed in the brain. It has the Genbank reference NP003387.1. Wnt16 has two isoforms, Wnt-16a and Wnt-16b, produced by alternative splicing. Isoform Wnt-16a is expressed in the pancreas. Isoform Wnt-16b is expressed in peripheral lymphoid organs such as spleen, appendix, and lymph nodes, or in the kidney, but not expressed in bone marrow. The Genbank references are NP476509.1 and NP057171.2, respectively, for Wnt16a and Wnt16b. All GenBank, SwissProt and other database sequences listed are expressly incorporated by reference herein.

A “variant” polypeptide means a biologically active polypeptide as defined below having less than 100% sequence identity with a native sequence polypeptide. Such variants include polypeptides wherein one or more amino acid residues are added at the N- or C terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid.

In some instances, a biologically active Wnt variant has an amino acid sequence having at least about 80% amino acid sequence identity with a native sequence Wnt polypeptide. In some instances, the biologically active Wnt variant has an amino acid sequence having at least about 85%, 90%, 91%, 92%, 93%, 94%, 95%, 96%, 96%, 97%, or 99% amino acid sequence identity with a native sequence Wnt polypeptide. In some cases, the biologically active Wnt variant has an amino acid sequence having at least about 95% amino acid sequence identity with a native sequence Wnt polypeptide. In some cases, the biologically active Wnt variant has an amino acid sequence having at least about 99% amino acid sequence identity with a native sequence Wnt polypeptide. In some embodiments, the biologically active Wnt variant is a Wnt3A variant. In some embodiments, the biologically active Wnt variant is a human Wnt3A variant.

In some instances, a biologically active Wnt variant comprises a lipid modification at one or more amino acid positions. In some cases, the lipid modification is at a position on a Wnt variant that is equivalent to position 77 set forth in SEQ ID NO: 1. In some cases, the lipid modification is at a position on a Wnt variant that is equivalent to position 209 set forth in SEQ ID NO: 1. In some cases, the lipid modification comprises both positions that are equivalent to positions 77 and 209 set forth in SEQ ID NO: 1. In some instances, the Wnt variant is Wnt3A, Wnt5A or Wnt 10B. In some cases, the Wnt variant is Wnt3A. In some cases, the Wnt3A variant comprises a lipid modification at a position equivalent to residue 77 set forth in SEQ ID NO: 1. In some cases, the Wnt3A variant comprises a lipid modification at a position equivalent to residue 209 set forth in SEQ ID NO: 1. In some cases, the Wnt3A variant comprises lipid modifications at positions equivalent to residues 77 and 209 set forth in SEQ ID NO: 1. In some cases, the modification is palmitoylation.

In some instances, a biologically active Wnt variant further comprises a residue modified by glycosylation. In some cases, the modification occurs at a position equivalent to position 82 and/or 298 set forth in SEQ ID NO: 1. In some cases, the Wnt variant is Wnt3A. In some cases, a Wnt3A variant further comprises a residue modified by glycosylation. In some cases, a Wnt3A variant further comprises a glycosylated residue at one or more positions equivalent to residue 82 and/or residue 298 set forth in SEQ ID NO: 1.

The term “amino acid” refers to a molecule containing both an amino group and a carboxyl group. Suitable amino acids include, without limitation, both the D- and L-isomers of the naturally-occurring amino acids, as well as non-naturally occurring amino acids prepared by organic synthesis or other metabolic routes. The term amino acid, as used herein, includes, without limitation, α-amino acids, natural amino acids, non-natural amino acids, and amino acid analogs.

The term “α-amino acid” refers to a molecule containing both an amino group and a carboxyl group bound to a carbon which is designated the α-carbon.

The term “β-amino acid” refers to a molecule containing both an amino group and a carboxyl group in a β configuration.

The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | 3- | 1- | Side- | Side-chain | |
|---|---|---|---|---|---|
| | Letter | Letter | chain | charge | Hydropathy |
| Amino Acid | Code | Code | Polarity | (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive(10%) | −3.2 |
| | | | | neutral(90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acid” are glycine, alanine, proline, and analogs thereof “Large hydrophobic amino acids” are valine, leucine, isoleucine, phenylalanine, methionine, tryptophan, and analogs thereof. “Polar amino acids” are serine, threonine, asparagine, glutamine, cysteine, tyrosine, and analogs thereof “Charged amino acids” are lysine, arginine, histidine, aspartate, glutamate, and analogs thereof.

The term “amino acid analog” refers to a molecule which is structurally similar to an amino acid and which can be substituted for an amino acid in the formation of a peptidomimetic macrocycle Amino acid analogs include, without limitation, β-amino acids and amino acids where the amino or carboxy group is substituted by a similarly reactive group (e.g., substitution of the primary amine with a secondary or tertiary amine, or substitution of the carboxy group with an ester).

The term “non-natural amino acid” refers to an amino acid which is not one of the twenty amino acids commonly found in peptides synthesized in nature, and known by the one letter abbreviations A, R, N, C, D, Q, E, G, H, I, L, K, M, F, P, S, T, W, Y and V. Non-natural amino acids or amino acid analogs include, without limitation, the following amino acid analogs.

Amino acid analogs include β-amino acid analogs. Examples of β-amino acid analogs include, but are not limited to, the following: cyclic β-amino acid analogs; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl) butyric acid; (S)-3-amino-4-(4-cyanophenyl)-butyric acid; (S)-3-amino-4-(4-fluorophenyl) butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogs include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; δ-chloro-L-alanine; β-cyano-L-alanin; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine-dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine.dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogs include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃)—OH; Nδ-benzyloxycarbonyl-L-ornithine; Nω-nitro-D-arginine; Nω-nitro-L-arginine; α-methyl-ornithine; 2,6-diaminoheptanedioic acid; L-ornithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-ornithine; (Nδ-4-methyltrityl)-D-ornithine; (Nδ-4-methyltrityl)-L-ornithine; D-ornithine; L-ornithine; Arg(Me)(Pbf)-OH; Arg(Me)₂-OH (asymmetrical); Arg(Me)2-OH (symmetrical); Lys(ivDde)-OH; Lys(Me)2-OH.HCl; Lys(Me3)-OH chloride; Nω-nitro-D-arginine; and Nω-nitro-L-arginine.

Amino acid analogs include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)-OH; and pyroglutamic acid.

Amino acid analogs include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Amino acid analogs include analogs of phenylalanine and tyrosine. Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogs include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

Amino acid analogs include analogs of serine and threonine. Examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogs include analogs of tryptophan. Examples of amino acid analogs of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some embodiments, amino acid analogs are racemic. In some embodiments, the D isomer of the amino acid analog is used. In some embodiments, the L isomer of the amino acid analog is used. In other embodiments, the amino acid analog comprises chiral centers that are in the R or S configuration. In still other embodiments, the amino group(s) of a β-amino acid analog is substituted with a protecting group, e.g., tert-butyloxycarbonyl (BOC group), 9-fluorenylmethyloxycarbonyl (FMOC), tosyl, and the like. In yet other embodiments, the carboxylic acid functional group of a β-amino acid analog is protected, e.g., as its ester derivative. In some embodiments the salt of the amino acid analog is used.

A “non-essential” amino acid residue is a residue that can be altered from the wild-type sequence of a polypeptide without abolishing or substantially altering its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, for example, is replaced with another amino acid residue from the same side chain family. Other examples of acceptable substitutions are substitutions based on isosteric considerations (e.g. norleucine for methionine) or other properties (e.g. 2-thienylalanine for phenylalanine, or 6-Cl-tryptophan for tryptophan).

### Biologically Active Wnt Polypeptide

Disclosed herein, in certain embodiments, are methods for generating substantially homogeneous biologically active Wnt compositions, which are purified from starting material secreted into minimal serum condition (e.g., serum-free medium). In some instances, described herein is an in vitro method of producing a biologically active Wnt polypeptide under a minimal serum condition, which comprises culturing cells from an engineered cell line transfected with an expression vector encoding a Wnt polypeptide under the minimal serum condition; and collecting secreted Wnt polypeptide from the culture medium under the minimal serum condition. In some cases, described herein also includes a culture medium which comprises minimal serum culture medium, a biologically active Wnt polypeptide secreted into the minimal serum culture medium, and cells from an engineered cell line transfected with an expression vector encoding the biologically active Wnt polypeptide, wherein the cells are grown in the presence of the minimal serum culture medium.

### Expression Construct

In some embodiments, a Wnt polypeptide comprising one or more variants is produced by recombinant methods. In some instances, the Wnt polypeptide is a Wnt3A, Wnt5A, or a wnt10b polypeptide. In some instances, the Wnt polypeptide comprising one or more variants is a Wnt3A polypeptide. In some instances, the Wnt polypeptide comprising one or more variants is a Wnt5A polypeptide. In some instances, the Wnt polypeptide comprising one or more variants is a Wnt10B polypeptide.

Amino acid sequence variants, including variants that are truncated at the C-terminus, are prepared by introducing appropriate nucleotide changes into the Wnt polypeptide DNA. Such variants represent insertions, substitutions, and/or specified deletions of, residues within or at one or both of the ends of the amino acid sequence of a naturally occurring Wnt polypeptide. Any combination of insertion, substitution, and/or specified deletion, e.g. truncation, is made to arrive at the final construct, provided that the final construct possesses the desired biological activity as defined herein. The amino acid changes also may alter post-translational processes of the Wnt polypeptide, such as changing the number or position of glycosylation sites, altering the membrane anchoring characteristics, and/or altering the intracellular location of the Wnt polypeptide by inserting, deleting, or otherwise affecting the leader sequence of the Wnt polypeptide.

In some embodiments, the one or more variants within a Wnt polypeptide comprise a substitution, insertion, deletion, or a combination thereof. In some instances, the Wnt3A polypeptide comprises a substitution, insertion, deletion, or a combination thereof. In some cases, the Wnt5A polypeptide comprises a substitution, insertion, deletion, or a combination thereof. In other cases, the Wnt10B polypeptide comprises a substitution, insertion, deletion, or a combination thereof.

In some cases, the DNA encoding a Wnt3A polypeptide is represented by SEQ ID NO:1 or SEQ ID NO: 2. In some cases, the DNA encoding a Wnt3A polypeptide is prepared, e.g. by truncating a sequence of SEQ ID NO:1, or by utilizing the sequence of SEQ ID NO:2. In some instances, the Wnt polypeptide-encoding gene is also obtained by oligonucleotide synthesis, amplification, etc. as known in the art.

The nucleic acid (e.g., cDNA or genomic DNA) encoding the Wnt polypeptide is inserted into a replicable vector for expression. Many such vectors are available. The vector components generally include, but are not limited to, one or more of the following: an origin of replication, one or more marker genes, an enhancer element, a promoter, and a transcription termination sequence. Preferably a GMP compatible vector is selected, for example the commercially available vectors OpticVec™ (expression vector), pTarget™ (expression vector), pcDNA™4 TO4 (expression vector), pcDNA™4.0 (expression vector), and the like.

In some embodiments, an expression vector that is tolerant of a minimal serum culture condition is used. In some instances, the minimal serum culture condition includes reduced-serum culture condition, protein-free culture condition, chemically defined media culture condition, or serum-free culture condition. In some embodiments, an expression vector that is tolerant of a reduced-serum culture condition is used. In some embodiments, an expression vector that is tolerant of a protein-free culture condition is used. In some embodiments, an expression vector that is tolerant of a chemically defined media culture condition is used.

In some embodiments, an expression vector that is tolerant of a serum-free medium condition is used. In some cases, the expression vector leads to a high copy number of the desired transcript and secretion of the protein of interest. In some instances, the expression vector is compatible with cGMP compatible mammalian cell lines. Non-limiting examples of mammalian expression vectors include pOptivec vector, plargeT™ vector, BacMam pCMV-Dest vector, Flp-In™ core system, Gateway® suite of vectors, HaloTag® vector, Flexi® vector, pCMVTNT™ vector, pcDNA™4.0, and pcDNA™4/TO vector. In some embodiments, the expression vector is selected from pOptivec and plargeT™ vectors. The pOptivec vector is a TOPO® adapted bicistronic plasmid which allows rapid cloning of a gene containing a mammalian secretion signal and the gene of interest downstream of the CMV promoter. The dihydrofolate reductase selection markers allows for rapid selection. In some cases, this vector is used for transient transfection of CHO-S cells. In some instances, the pTargeT™ vector is used for transient transfection of CHO-S cells and for creating a stable cell line expressing a Wnt protein (e.g. Wnt3A).

The coding sequence will also include a signal sequence that allows secretion of the WNT. The signal sequence may be a component of the vector, or it may be a part of the Wnt encoding DNA that is inserted into the vector. A heterologous signal sequence selected preferably is one that is recognized and processed (i.e., cleaved by a signal peptidase) by the host cell. In mammalian cell expression the native signal sequence may be used, or other mammalian signal sequences may be suitable, such as signal sequences from other animal Wnt polypeptide, and signal sequences from secreted polypeptides of the same or related species, as well as viral secretory leaders, for example, the herpes simplex gD signal.

Expression vectors may contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors will contain a promoter that is recognized by the host organism and is operably linked to the Wnt coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known. Both a native Wnt polypeptide promoter sequence and many heterologous promoters may be used to direct expression of a Wnt polypeptide. However, heterologous promoters are preferred, as they generally permit greater transcription and higher yields.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment.

Transcription may be increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors used in mammalian host cells will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding Wnt polypeptide.

Construction of suitable vectors containing one or more of the above-listed components employs standard techniques. Isolated plasmids or DNA fragments are cleaved, tailored, and re-ligated in the form desired to generate the vectors required.

Particularly useful in the practice of this invention are expression vectors that provide for the transient expression in mammalian cells. In general, transient expression involves the use of an expression vector that is able to replicate efficiently in a host cell, such that the host cell accumulates many copies of the expression vector and, in turn, synthesizes high levels of a desired polypeptide encoded by the expression vector. Transient expression systems, comprising a suitable expression vector and a host cell, allow for the convenient positive identification of polypeptides encoded by cloned DNAs, as well as for the rapid screening of such polypeptides for desired biological or physiological properties.

In some instances, serum-free media is used. Non-limiting examples of serum-free media include CD CHO medium, CD CHO AGT™ medium, CD OptiCHO™ medium, CHO-S-SFM II (optionally including hypoxanthine and thymidine), CD 293 AGT™ medium, Adenovirus Expression Medium (AEM), FreeStyle™ 293 Expression medium, FreeStyle™ CHO Expression medium, CD FortiCHO™ medium, EX-CELL® 302 Serum-Free medium, EX-CELL® 325 PF CHO Serum-Free medium, EX-CELL® CD CHO-2 medium animal-component free, EX-CELL® CD CHO-3 medium, and EX-CELL® CDHO DHFR⁻ medium animal-component free.

The methods of the present invention may be performed so as to conform with FDA or WHO guidelines for GMP production. Guidelines for such may be obtained from the relevant regulatory agency. See, for example, “WHO good manufacturing practices: main principles for pharmaceutical products. Annex 3 in: WHO Expert Committee on Specifications for Pharmaceutical Preparations. Forty-fifth report. Geneva, World Health Organization, 2011 (WHO Technical Report Series, No. 961)”; “ICH Q5B guideline. Analysis of the expression construct in cells used for production of r-DNA derived protein products. Geneva, International Conference on Harmonisation of Technical Requirements for Registration of Pharmaceuticals for Human Use, 1995”; “Handbook: good laboratory practice (GLP): quality practices for regulated non-clinical research and development, 2nd ed. Geneva, UNDP/World Bank/WHO, Special Programme for Research and Training in Tropical Diseases, 2009”; each herein specifically incorporated by reference.

Typically, recombinant DNA-derived biotherapeutics are produced using a cell bank system which involves a manufacturer's working cell bank (WCB) derived from a master cell bank. The present invention includes frozen aliquots of CHO-S cells transfected with a vector for secretion of the WNT3A protein, which cells can be used as a master cell bank or as a working cell bank.

### Methods

Disclosed herein include the development of a serum-free process for the secretion of biologically active Wnt polypeptide. In some instances, the biologically active Wnt polypeptide is a human biologically active Wnt polypeptide. In some cases, the biologically active Wnt polypeptide is a Wnt3A, Wnt5A, or Wnt10B polypeptide. In some cases, the biologically active Wnt polypeptide is a Wnt3A polypeptide. In some cases, the biologically active Wnt polypeptide is human Wnt3A polypeptide.

In some embodiments, a cGMP compatible cell line is transfected with an expression vector encoding a Wnt polypeptide. Exemplary cGMP compatible cell line includes mammalian cell lines such as Chinese Hamster Ovary (CHO) cell line, human embryonic kidney (HEK) cell line, or baby hamster kidney (BHK) cell line; or insect cell lines such as Sf9 cell line, Sf21 cell line, Tn-368 cell line, or High Five (BTI-TN-5B1-4) cell line.

In some instances, an expression vector encoding a Wnt polypeptide is transfected in a cGMP compatible cell line selected from Chinese Hamster Ovary (CHO) cell line, human embryonic kidney (HEK) cell line, baby hamster kidney (BHK) cell line, Sf9 cell line, Sf21 cell line, Tn-368 cell line, or High Five (BTI-TN-5B1-4) cell line. In some instances, an expression vector encoding a Wnt polypeptide is transfected in a CHO cell line. In some instances, an expression vector encoding a Wnt polypeptide is transfected in a BHK cell line. In some instances, an expression vector encoding a Wnt polypeptide is transfected in a HEK cell line. In some instances, an expression vector encoding a Wnt polypeptide is transfected in a Sf9 cell line. In some instances, an expression vector encoding a Wnt polypeptide is transfected in a Sf21 cell line. In some instances, an expression vector encoding a Wnt polypeptide is transfected in a Tn-368 cell line. In some instances, an expression vector encoding a Wnt polypeptide is transfected in a High Five cell line. In some cases, the Wnt polypeptide is Wnt3A polypeptide, Wnt 5a polypeptide, or Wnt 10b polypeptide.

In some embodiments, the Wnt polypeptide is Wnt3A polypeptide. In some instances, an expression vector encoding Wnt3A polypeptide is transfected in a cGMP compatible cell line selected from Chinese Hamster Ovary (CHO) cell line, human embryonic kidney (HEK) cell line, baby hamster kidney (BHK) cell line, Sf9 cell line, Sf21 cell line, Tn-368 cell line, or High Five (BTI-TN-5B1-4) cell line. In some instances, an expression vector encoding Wnt3A polypeptide is transfected in a CHO cell line. In some instances, an expression vector encoding Wnt3A polypeptide is transfected in a BHK cell line. In some instances, an expression vector encoding Wnt3A polypeptide is transfected in a HEK cell line. In some instances, an expression vector encoding Wnt3A polypeptide is transfected in a Sf9 cell line. In some instances, an expression vector encoding Wnt3A polypeptide is transfected in a Sf21 cell line. In some instances, an expression vector encoding Wnt3A polypeptide is transfected in a Tn-368 cell line. In some instances, an expression vector encoding Wnt3A polypeptide is transfected in a High Five cell line.

In some cases, the CHO cell line is CHO-S cell line. In some instances, an expression vector encoding a Wnt polypeptide is transfected in CHO-S cell line. In some cases, the Wnt polypeptide is Wnt3A polypeptide, Wnt 5a polypeptide, or Wnt 10b polypeptide. In some instances, an expression vector encoding Wnt3A polypeptide is transfected in CHO-S cell line. In some cases, an expression vector encoding SEQ ID NO: 1 or SEQ ID NO: 2 of Wnt3A polypeptide is transfected in CHO-S cell line. In additional cases, an expression vector encoding a Wnt3A polypeptide comprising a variant (e.g., a deletion or truncation) is transfected in CHO-S cell line.

In some instances, the combination of CHO-S cells transfected with an expression vector encoding Wnt3A polypeptide comprising a deletion or a truncation allows effective secretion of the protein into minimal serum culture medium (e.g., serum-free condition). In some cases, the deletion or truncation is a C-terminus deletion or truncation. In some instances, the Wnt3A polypeptide is as illustrated in SEQ ID NO: 1. In some cases, the combination of CHO-S cells transfected with an expression vector encoding Wnt3A polypeptide in which, relative to SEQ ID NO:1 (BC103921), the C-terminus is truncated, allows effective secretion of the protein into culture medium in the absence of serum or other animal products.

As described elsewhere herein, the minimal serum medium sometimes comprises less than 9% serum. In some cases, the serum is FBS. In some cases, the FBS presents in the minimal serum medium is at most about 0.05%, 0.1%, 0.5%, 1%, 2%, 3%, 4%, 5%, 6%, 7%, 8%, 9%, or less. In some cases, the FBS presents in the minimal serum medium is at least about 0.05%, 0.1%, 0.5%, 1%, 2%, 3%, 4%, 5%, 6%, 7%, 8%, 9%, or more. In some cases, the FBS presents in the minimal serum medium is about 0.05%. In some cases, the FBS presents in the minimal serum medium is about 0.1%. In some cases, the FBS presents in the minimal serum medium is about 0.5%. In some cases, the FBS presents in the minimal serum medium is about 1%. In some cases, the FBS presents in the minimal serum medium is about 2%. In some cases, the FBS presents in the minimal serum medium is about 3%. In some cases, the FBS presents in the minimal serum medium is about 4%. In some cases, the FBS presents in the minimal serum medium is about 5%. In some cases, the FBS presents in the minimal serum medium is about 6%. In some cases, the FBS presents in the minimal serum medium is about 7%. In some cases, the FBS presents in the minimal serum medium is about 8%. In some cases, the FBS presents in the minimal serum medium is about 9%. In other cases, the minimal serum medium is a serum-free medium.

Sometimes, the minimal serum medium comprises components such as peptides and/or polypeptides obtained from plant hydrolysates but not proteins or components of animal origin. In other cases, the minimal serum medium comprises recombinant proteins and/or hormones and does not comprise FBS, bovine serum albumin, or human serum albumin. In additional cases, the minimal serum medium comprises low molecular weight constituents and optionally synthetic peptides and/or hormones.

In some embodiments, the minimal serum medium contains one or more additional supplement. In some embodiments, the additional supplement is a lipid supplement. Non-limiting examples of lipid supplement include Lipid Mixture 1 (Sigma-Aldrich), Lipid Mixture 2 (Sigma-Aldrich), Lipogro® a bovine cholesterol concentrate (Rocky Mountain Biologicals), and Chemically Defined Lipid Concentration (Life Technologies). In some embodiments, the serum-free medium contains a lipid supplement.

In some embodiments, the methods of the invention comprise culturing in serum-free medium CHO cells (e.g., CHO-S cells) transfected with an expression vector comprising a C-terminal truncated Wnt polypeptide (e.g., Wnt3A polypeptide) comprising a signal sequence for secretion, which can be the native Wnt (e.g., Wnt3A) signal sequence or a heterologous signal sequence, operably linked to a promoter, under conditions in which the Wnt polypeptide (e.g., Wnt3A polypeptide) is expressed and secreted. In some embodiments, the methods further comprise an initial step of transfecting the cells with the expression vector. In some embodiment the methods comprise purifying the polypeptide thus produced from the medium. In some embodiments the Wnt polypeptide (e.g., Wnt3A polypeptide) is purified to a degree suitable for GMP clinical use. In some embodiments the Wnt polypeptide (e.g., Wnt3A polypeptide) thus purified is packaged in a unit dose formulation.

In some embodiments the CHO cells are grown in suspension. In some embodiments the CHO cells are adherent. In some embodiments the medium comprises a serum substitute. In some embodiments the serum substitute is free of animal products. In some embodiments the serum substitute comprises purified proteins, e.g. one or more of insulin, transferrin, bovine serum albumin, human serum albumin, etc., but which lacks, for example, growth factors, steroid hormones, glucocorticoids, cell adhesion factors, detectable Ig, mitogens, etc. The serum substitute may be present at a concentration in the medium of up to about 0.1%, up to about 0.25%, up to about 0.5%, up to about 0.75%, up to about 1%, up to about 2.5%, up to about 5%, up to about 7.5%, or up to about 10%. The serum substitute may be present at a concentration in the medium of up to about 0.1%. The serum substitute may be present at a concentration in the medium of up to about 0.25%. The serum substitute may be present at a concentration in the medium of up to about 0.5%. The serum substitute may be present at a concentration in the medium of up to about 0.75%. The serum substitute may be present at a concentration in the medium of up to about 1%. The serum substitute may be present at a concentration in the medium of up to about 2.5%. The serum substitute may be present at a concentration in the medium of up to about 5%. The serum substitute may be present at a concentration in the medium of up to about 7.5%. The serum substitute may be present at a concentration in the medium of up to about 10%.

Suitable medium may be selected from those known in the art, including without limitation DMEM, RPMI-1640, MEM, Iscove's, CHO Cell Medium; and the like. Suitable serum substitutes include those produced with no animal products, or those with only purified animal protein components. Commercially available supplements suitable for this purpose include, without limitation, CellEsse, ITS (Insulin-Transferrin-Selenium) (e.g., ITS3 or ITS3+), Excyte®, OneShot™ (One Shot fetal bovine serum), Knockout™ Media, and the like as known in the art. In some instances, the ITS (Insulin-Transferrin-Selenium) supplement is a supplement comprising a mixture of insulin, transferrin, and selenium. The medium may further comprise, without limitation, such components as GlutaMax™ (a glutamine-based dipeptide), antibiotic (e.g. doxycycline), G418, non-essential amino acids, blasticidine, etc.

The level of secretion of the Wnt polypeptide into the serum-free culture medium may be at least about 10 ng/ml, at least about 25 ng/ml, at least about 50 ng/ml, at least about 75 ng/ml, at least about 100 ng/ml, at least about 250 ng/ml, at least about 500 ng/ml, at least about 750 ng/ml, at least about 1 μg/ml, at least about 1.1 μg/ml, at least about 1.25 μg/ml, at least about 1.5 μl/ml, at least about 1.75 μl/ml, at least about 2.5 μl/ml, at least about 5 μg/ml, at least about 7.5 μl/ml, at least about 10 μg/ml or more. The level of secretion of the Wnt polypeptide into the serum-free culture medium may be at least about 10 ng/ml. The level of secretion of the Wnt polypeptide into the serum-free culture medium may be at least about 25 ng/ml. The level of secretion of the Wnt polypeptide into the serum-free culture medium may be at least about 50 ng/ml. The level of secretion of the Wnt polypeptide into the serum-free culture medium may be at least about 75 ng/ml. The level of secretion of the Wnt polypeptide into the serum-free culture medium may be at least about 100 ng/ml. The level of secretion of the Wnt polypeptide into the serum-free culture medium may be at least about 250 ng/ml. The level of secretion of the Wnt polypeptide into the serum-free culture medium may be at least about 500 ng/ml. The level of secretion of the Wnt polypeptide into the serum-free culture medium may be at least about 750 ng/ml. The level of secretion of the Wnt polypeptide into the serum-free culture medium may be at least about 1 μg/ml. The level of secretion of the Wnt polypeptide into the serum-free culture medium may be at least about 1.1 μg/ml. The level of secretion of the Wnt polypeptide into the serum-free culture medium may be at least about 1.25 μg/ml. The level of secretion of the Wnt polypeptide into the serum-free culture medium may be at least about 1.5 μg/ml. The level of secretion of the Wnt polypeptide into the serum-free culture medium may be at least about 1.75 μg/ml. The level of secretion of the Wnt polypeptide into the serum-free culture medium may be at least about 2.5 μg/ml. The level of secretion of the Wnt polypeptide into the serum-free culture medium may be at least about 5 μg/ml. The level of secretion of the Wnt polypeptide into the serum-free culture medium may be at least about 7.5 μg/ml. The level of secretion of the Wnt polypeptide into the serum-free culture medium may be at least about 10 μg/ml. In some instances, the Wnt polypeptide is Wnt3A polypeptide. In some cases, the Wnt polypeptide is Wnt5A polypeptide. In some cases, the Wnt polypeptide is Wnt 10B polypeptide.

In some instances, the Wnt polypeptide is Wnt3A polypeptide. In some cases, the level of secretion of the Wnt3A polypeptide into the serum-free culture medium is at least about 10 ng/ml, at least about 25 ng/ml, at least about 50 ng/ml, at least about 75 ng/ml, at least about 100 ng/ml, at least about 250 ng/ml, at least about 500 ng/ml, at least about 750 ng/ml, at least about 1 μg/ml, at least about 1.1 μg/ml, at least about 1.25 μg/ml, at least about 1.5 μg/ml, at least about 1.75 μl/ml, at least about 2.5 μl/ml, at least about 5 μl/ml, at least about 7.5 μl/ml, at least about 10 μg/ml or more.

The level of secretion of the Wnt3A polypeptide into the serum-free culture medium may be at least about 10 ng/ml. The level of secretion of the Wnt3A polypeptide into the serum-free culture medium may be at least about 25 ng/ml. The level of secretion of the Wnt3A polypeptide into the serum-free culture medium may be at least about 50 ng/ml. The level of secretion of the Wnt3A polypeptide into the serum-free culture medium may be at least about 75 ng/ml. The level of secretion of the Wnt3A polypeptide into the serum-free culture medium may be at least about 100 ng/ml. The level of secretion of the Wnt3A polypeptide into the serum-free culture medium may be at least about 250 ng/ml. The level of secretion of the Wnt3A polypeptide into the serum-free culture medium may be at least about 500 ng/ml. The level of secretion of the Wnt3A polypeptide into the serum-free culture medium may be at least about 750 ng/ml. The level of secretion of the Wnt3A polypeptide into the serum-free culture medium may be at least about 1 μg/ml. The level of secretion of the Wnt3A polypeptide into the serum-free culture medium may be at least about 1.1 μg/ml. The level of secretion of the Wnt3A polypeptide into the serum-free culture medium may be at least about 1.25 μg/ml. The level of secretion of the Wnt3A polypeptide into the serum-free culture medium may be at least about 1.5 μg/ml. The level of secretion of the Wnt3A polypeptide into the serum-free culture medium may be at least about 1.75 μg/ml. The level of secretion of the Wnt3A polypeptide into the serum-free culture medium may be at least about 2.5 μg/ml. The level of secretion of the Wnt3A polypeptide into the serum-free culture medium may be at least about 5 μg/ml. The level of secretion of the Wnt3A polypeptide into the serum-free culture medium may be at least about 7.5 μg/ml. The level of secretion of the Wnt3A polypeptide into the serum-free culture medium may be at least about 10 μg/ml.

In some embodiments, the C-terminus of the expressed and secreted Wnt polypeptide is truncated by between 5 to 40 amino acids. In some instances, the C-terminus of the expressed and secreted Wnt polypeptide is truncated by between 5 to 35 amino acids, between 10 to 35 amino acids, between 10 to 33 amino acids, between 10 to 30 amino acids, between 15 to 33 amino acids, between 15 to 30 amino acids, between 20 to 35 amino acids, between 20 to 33 amino acids, between 20 to 30 amino acids, between 25 to 33 amino acids or between 25 to 30 amino acids.

In some embodiments, the C-terminus of the expressed and secreted Wnt polypeptide is truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or more amino acids, and may be additionally truncated at the N or C terminus, provided that the protein maintains biological activity. In some embodiments the Wnt polypeptide is truncated by 5 amino acids. In some embodiments the Wnt polypeptide is truncated by 10 amino acids. In some embodiments the Wnt polypeptide is truncated by 15 amino acids. In some embodiments the Wnt polypeptide is truncated by 20 amino acids. In some embodiments the Wnt polypeptide is truncated by 25 amino acids. In some embodiments the Wnt polypeptide is truncated by 30 amino acids. In some embodiments the Wnt polypeptide is truncated by 33 amino acids.

In some instances, the Wnt polypeptide is Wnt3A polypeptide. In some embodiments, the C-terminus of the expressed and secreted Wnt3A polypeptide is truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or more amino acids, and may be additionally truncated at the N or C terminus, provided that the protein maintains biological activity. In some embodiments the Wnt3A polypeptide is truncated by 5 amino acids. In some embodiments the Wnt3A polypeptide is truncated by 10 amino acids. In some embodiments the Wnt3A polypeptide is truncated by 15 amino acids. In some embodiments the Wnt3A polypeptide is truncated by 20 amino acids. In some embodiments the Wnt3A polypeptide is truncated by 25 amino acids. In some embodiments the Wnt3A polypeptide is truncated by 30 amino acids. In some embodiments the Wnt3A polypeptide is truncated by 33 amino acids.

In some embodiments, the Wnt3A polypeptide has a sequence of at least 70%, 80%, 85%, 90%, 95%, 96%, 97%, 98%, or 99% sequence identity to SEQ ID NO:1. In some embodiments, the Wnt3A polypeptide has a sequence of at least 70% sequence identity to SEQ ID NO:1. In some embodiments, the Wnt3A polypeptide has a sequence of at least 80% sequence identity to SEQ ID NO:1. In some embodiments, the Wnt3A polypeptide has a sequence of at least 85% sequence identity to SEQ ID NO:1. In some embodiments, the Wnt3A polypeptide has a sequence of at least 90% sequence identity to SEQ ID NO:1. In some embodiments, the Wnt3A polypeptide has a sequence of at least 95% sequence identity to SEQ ID NO:1. In some embodiments, the Wnt3A polypeptide has a sequence of at least 96% sequence identity to SEQ ID NO:1. In some embodiments, the Wnt3A polypeptide has a sequence of at least 97% sequence identity to SEQ ID NO:1. In some embodiments, the Wnt3A polypeptide has a sequence of at least 98% sequence identity to SEQ ID NO:1. In some embodiments, the Wnt3A polypeptide has a sequence of at least 99% sequence identity to SEQ ID NO:1.

In some embodiments the Wnt3A polypeptide has a sequence of at least 70%, 80%, 85%, 90%, 95%, 96%, 97%, 98%, or 99% sequence identity to SEQ ID NO:2. In some embodiments, the Wnt3A polypeptide has a sequence of at least 70% sequence identity to SEQ ID NO:2. In some embodiments, the Wnt3A polypeptide has a sequence of at least 80% sequence identity to SEQ ID NO:2. In some embodiments, the Wnt3A polypeptide has a sequence of at least 85% sequence identity to SEQ ID NO:2. In some embodiments, the Wnt3A polypeptide has a sequence of at least 90% sequence identity to SEQ ID NO:2. In some embodiments, the Wnt3A polypeptide has a sequence of at least 95% sequence identity to SEQ ID NO:2. In some embodiments, the Wnt3A polypeptide has a sequence of at least 96% sequence identity to SEQ ID NO:2. In some embodiments, the Wnt3A polypeptide has a sequence of at least 97% sequence identity to SEQ ID NO:2. In some embodiments, the Wnt3A polypeptide has a sequence of at least 98% sequence identity to SEQ ID NO:2. In some embodiments, the Wnt3A polypeptide has a sequence of at least 99% sequence identity to SEQ ID NO:2.

In some embodiments the Wnt polypeptide (e.g., Wnt3A polypeptide) is purified to an initial concentration of at least about 5 μg/ml; usually at least about 10 μg/ml, more usually at least about 50 μg/ml, and may be present at greater than about 100 μg/ml. The Wnt polypeptide (e.g., Wnt3A polypeptide) may be formulated in a liposome. The Wnt polypeptide (e.g., Wnt3A polypeptide) may be stabilized in a formulation with a detergent. The Wnt polypeptide (e.g., Wnt3A polypeptide) may be stabilized in a formulation with lipids.

In some embodiments, the liposome is fabricated using methods well known in the art. Liposomes are artificially-prepared spherical vesicles that compose a lamellar phase lipid bilayer and an aqueous core. There are several types of liposomes, such as the multilamellar vesicle (MLV), small unilamellar liposome vesicle (SUV), the large unilamellar vesicle (LUV), and the cochleate vesicle. In some instances, liposomes are formed by phospholipids. In some embodiments, phospholipids are separated into those with diacylglyceride structures or those derived from phosphosphingolipids. In some embodiments, the diacylglyceride structures include phosphatidic acid (phosphatidate) (PA), phosphatidylethanolamine (cephalin) (PE), phosphatidylcholine (lecithin) (PC), phosphatidylserine (PS), and phosphoinositides such as phosphatidylinositol (PI), phosphatidylinositol phosphate (PIP), phosphatidylinositol bisphosphate (PIP2), and phosphatidylinositol triphosphate (PIP3). In some embodiments, phosphosphingolipids include ceramide phosphorylcholine, ceramide phosphorylethanolamine, and ceramide phosphoryllipid. In some embodiments, the liposomes are formed from phosphatidylcholines.

In some embodiments, the lipids are also selected based on its transition phase temperature (Tₘ), or the temperature interface between the liquid crystalline phase and the gel phase. In some embodiments, the Tₘ is governed by the head group species, hydrocarbone length, unsaturation, and the charge. For example, short lipids (lipids containing 8, 10, or 12 tail carbon chain length) have liquid crystalline phase at temperatures below 4° C. However, liposomes manufactured from these short chain carbon lipids are toxic to cells because they dissolve cell membranes. Liposomes manufactured from longer carbon-chain lipids are not toxic to cells, but their transition temperatures are higher. For example, 1,2-dipalmitoyl-sn-glycero-3-phosphocholine (DPPC) which has a 16 tail carbon length, has a T, of about 41° C. In some embodiments, the lipids used herein have a T, of between about 10° C. and about 37° C., 15° C. and about 30° C., 18° C. and about 27° C., or 21° C. and about 25° C. In some embodiments, the lipids used herein have a Tₘ of at least 22° C., 23° C., 24° C., or more. In some embodiments, the lipids used herein have a Tₘ of at most 22° C., 23° C., 24° C., or less. In some embodiments, the lipids used herein have a tail carbon length of at least about 12, 13, 14, or more. In some embodiments, the lipids used herein have a tail carbon length of at most about 12, 13, 14, or less.

In some embodiments, the lipids are further selected based on the net charge of the liposome. In some embodiments, the liposome has a net charge of 0 at a pH of between about 4.0 and about 10.0, about 5.0 and about 9.0, about 6.5 and about 8.0, about 7.0 and about 7.8, or about 7.2 and about 7.6. In some embodiments, the liposome has a net charge of 0 at a pH of about 7.3, about 7.4, or about 7.5. In some embodiments, the liposome has a net positive charge at a pH of between about 4.0 and about 10.0, about 5.0 and about 9.0, about 6.5 and about 8.0, about 7.0 and about 7.8, or about 7.2 and about 7.6. In some embodiments, the liposome has a net positive charge at a pH of about 7.3, about 7.4, or about 7.5. In some embodiments, the liposome has a net negative charge at a pH of between about 4.0 and about 10.0, about 5.0 and about 9.0, about 6.5 and about 8.0, about 7.0 and about 7.8, or about 7.2 and about 7.6. In some embodiments, the liposome has a net negative charge at a pH of about 7.3, about 7.4, or about 7.5.

In some embodiments, lipids are selected from 1,2-dimyristoyl-sn-glycero-3-phosphocholine (DMPC), 1,2-dipalmitoyl-sn-glycero-3-phosphocholine (DPPC), 1-tetradecanoyl-2-hexadecanoyl-sn-glycero-3-phosphocholine (MPPC), 1,2-dimyristoyl-sn-glycero-3-phospho-L-serine (DMPS), and 1,2-dihexanoyl-sn-glycero-3-phosphocholine (DMPG). In some embodiments, the lipid is DMPC.

In some embodiments, an additional lipid is fabricated into the liposome. In some embodiments, the additional lipid is cholesterol. In some instances, the concentration of a phosphatidylcholine such as DMPC and cholesterol is defined by a value such as a ratio. In some embodiments, the ratio of the concentrations of phosphatidylcholine such as DMPC and cholesterol is between about 50:50, about 55:45, about 60:40, about 65:35, about 70:30, about 75:25, about 80:20, about 85:15, about 90:10, about 95:5, about 99:1, or about 100:0. In some embodiments, the ratio of the concentrations of phosphatidylcholine such as DMPC and cholesterol is about 90:10. In some embodiments, the concentration unit is moles. In some embodiments, the ratio is mole:mole.

In some embodiments, the Wnt polypeptide is reconstituted with a liposome at a concentration of at least about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.4, 0.5 ng/μL or more. In some embodiments, the Wnt polypeptide is reconstituted with a liposome at a concentration of at most about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.4, 0.5 ng/μL or less. In some embodiments, the Wnt polypeptide is Wnt3A polypeptide, Wnt5A polypeptide, or Wnt10b polypeptide. In some embodiments, the Wnt polypeptide is Wnt3A polypeptide.

In some embodiments, the Wnt polypeptide is reconstituted with a liposome at a ratio of at least about 0.1:50, 0.5:30, 1:20, or 1:14 Wnt polypeptide to liposome, or more. In some embodiments, the Wnt polypeptide is reconstituted with a liposome at a ratio of at most about 0.1:50, 0.5:30, 1:20, or 1:14 Wnt polypeptide to liposome, or less. In some instances, the ratio is a weight to weight ratio. In some instances, the unit of Wnt polypeptide is nanogram unit.

In some embodiments, the temperature at which the Wnt polypeptide is reconstituted with a liposome is at least between about 15° C. and about 37° C., about 18° C. and about 33° C., or about 20° C. and about 28° C. In some embodiments, the temperature is at least about 21° C., 22° C., 23° C., 24° C., 25° C., 26° C., 27° C., or more. In some embodiments, the temperature is at most about 21° C., 22° C., 23° C., 24° C., 25° C., 26° C., 27° C., or less. In some embodiments, the Wnt polypeptide is Wnt3A polypeptide, Wnt5A polypeptide, or Wnt10b polypeptide. In some embodiments, the Wnt polypeptide is Wnt3A polypeptide.

In some embodiments, the Wnt polypeptide is integrated into the liposomal membrane. In some cases, the Wnt polypeptide protrudes from the liposomal membrane onto the surface of the lipid membrane. In some instances, the Wnt polypeptide is not incorporated into the aqueous core of the liposome. In some embodiments, the Wnt polypeptide is Wnt3A polypeptide, Wnt5A polypeptide, or Wnt10B polypeptide. In some embodiments, the Wnt polypeptide is Wnt3A polypeptide. In some embodiments, the Wnt3A polypeptide is integrated into the liposomal membrane. In some cases, the Wnt3A polypeptide protrudes from the liposomal membrane onto the surface of the lipid membrane. In some instances, the Wnt3A polypeptide is not incorporated into the aqueous core of the liposome.

In some embodiments, the Wnt polypeptide reconstituted with a liposome is referred to as liposomal Wnt polypeptide or L-Wnt. In some embodiments, the Wnt polypeptide is Wnt3A polypeptide, Wnt5A polypeptide, or Wnt10B polypeptide. In some embodiments, the Wnt polypeptide is Wnt3A polypeptide. In some embodiments, the Wnt3A polypeptide reconstituted with a liposome is referred to as liposomal Wnt3A polypeptide or L-Wnt3A. In some embodiments, the Wnt polypeptide is Wnt5A polypeptide. In some embodiments, the Wnt5A polypeptide reconstituted with a liposome is referred to as liposomal Wnt5A polypeptide or L-Wnt5A. In some embodiments, the Wnt polypeptide is Wnt10B polypeptide. In some embodiments, the Wnt10B polypeptide reconstituted with a liposome is referred to as liposomal Wnt10B polypeptide or L-Wnt10B.

In some embodiments, the L-Wnt undergoes a centrifugation step and is then suspended in a buffer such as phosphate buffered saline (PBS). In some instances, the L-Wnt is stored under nitrogen. In some instances, the L-Wnt is stable under nitrogen without substantial loss of activity. In some instances, the L-Wnt is stored at a temperature of between about 1° C. and about 8° C. In some instances, the L-Wnt is stable at a temperature of at least about 1° C., 2° C., 3° C., 4° C., 5° C., 6° C., 7° C., 8° C., or more without substantial loss of activity. In some instances, the L-Wnt is stable at a temperature of at most about 1° C., 2° C., 3° C., 4° C., 5° C., 6° C., 7° C., 8° C., or less without substantial loss of activity. In some embodiments, the L-Wnt is stable for at least about 10, 15, 20, 25, 30, 35, 40, 45, 50, 55, 60, 65, 70, 75, 80, 85, 90, 95, 100, 101, 102, 103, 104, 105, 106, 107, 108, 109, 110, 115, 120, 130, 140, 150, 160, 170, 180, 190, 200, 300, 356, 400, 700, 1000 days, or more without substantial loss of activity. In some embodiments, the L-Wnt is stable for at most about 10, 15, 20, 25, 30, 35, 40, 45, 50, 55, 60, 65, 70, 75, 80, 85, 90, 95, 100, 101, 102, 103, 104, 105, 106, 107, 108, 109, 110, 115, 120, 130, 140, 150, 160, 170, 180, 190, 200, 300, 356, 400, 700, 1000 days, or less without substantial loss of activity.

In some embodiments, the L-Wnt3A undergoes a centrifugation step and is then suspended in a buffer such as phosphate buffered saline (PBS). In some instances, the L-Wnt3A is stored under nitrogen. In some instances, the L-Wnt3A is stable under nitrogen without substantial loss of activity. In some instances, the L-Wnt3A is stored at a temperature of between about 1° C. and about 8° C. In some instances, the L-Wnt3A is stable at a temperature of at least about 1° C., 2° C., 3° C., 4° C., 5° C., 6° C., 7° C., 8° C., or more without substantial loss of activity. In some instances, the L-Wnt3A is stable at a temperature of at most about 1° C., 2° C., 3° C., 4° C., 5° C., 6° C., 7° C., 8° C., or less without substantial loss of activity. In some embodiments, the L-Wnt3A is stable for at least about 10, 20, 30, 40, 50, 60, 70, 80 90, 100, 110, 120, 130, 140, 150, 160, 170, 180, 190, 200, 300, 356, 400, 700, 1000 days, or more without substantial loss of activity. In some embodiments, the L-Wnt3A is stable for at most about 10, 20, 30, 40, 50, 60, 70, 80 90, 100, 110, 120, 130, 140, 150, 160, 170, 180, 190, 200, 300, 356, 400, 700, 1000 days, or less without substantial loss of activity.

In some instances, the term “without substantial loss of activity” refers to the functional activity of a liposomal Wnt polypeptide is near to that of the corresponding native Wnt polypeptide in the absence of a liposome. In some instances, the functional activity of the liposomal Wnt polypeptide is at least about 100%, 99%, 95%, 90%, 85%, 80%, 75%, 70%, 60%, 50%, 40%, or more compared to the functional activity of the native Wnt polypeptide. In some instances, the functional activity of the liposomal Wnt polypeptide is at most about 100%, 99%, 95%, 90%, 85%, 80%, 75%, 70%, 60%, 50%, 40%, or less compared to the functional activity of the native Wnt polypeptide. In some instances, the functional activity of the Wnt polypeptides is detected using assays such as for example mass spectroscopy, assays associated with biomarker analysis which are described elsewhere herein, transplant surgery such as sub-renal capsule transplant surgery, spinal fusion surgery, ALP, TRAP, and TUNEL staining, immunohistochemistry, and Micro-CT analyses and quantification of graft growth.

In some instances, the term “stable” refers to Wnt polypeptides as in a folded state and is not unfolded or degraded. In some instances, the term “stable” also refers to Wnt polypeptides retaining functional activity without substantial loss of activity. In some instances, assays used to determine stability assays that establish the activity of the Wnt polypeptides, as such those described above, and also include such as LSL cell-based assays such as mice LSL cell-based assay.

In some embodiments, disclosed herein is a method of preparing a liposomal Wnt polypeptide with use of a chaperone. In some instances, the method comprises (a) incubating an isolated Wnt polypeptide with a plurality of chaperones to generate a Wnt polypeptide-chaperone complex; (b) separating the Wnt polypeptide-chaperone complex from non-complexed chaperones; and (c) contacting the Wnt polypeptide-chaperone complex with an aqueous solution of liposomes to generate the liposomal Wnt polypeptide.

In some instances, also disclosed herein is a method of purifying Wnt polypeptides with use of a chaperone. In some instances, the method comprises (a) incubating a liposomal Wnt polypeptide with a plurality of chaperones to form a liposomal Wnt polypeptide-chaperone complex; and (b) separating the liposomal Wnt polypeptide-chaperone complex from non-complexed chaperones to generate purified liposomal Wnt polypeptides; and (c) eluting the liposomal Wnt polypeptide from the liposomal Wnt polypeptide-chaperone complex to generate a purified liposomal Wnt polypeptide.

In some instances, a chaperone described herein comprises a protein or fragments thereof that facilitates in the assembly or disassembly of a macromolecular structure. In some instances, a chaperone comprises a protein or fragments thereof that facilitates in a purification method. As used herein in the context of Wnt polypeptides, a chaperone comprises a protein or fragments thereof that facilitates in purification of isolated Wnt polypeptides and/or preparation of a liposomal Wnt polypeptide. Furthermore, as used herein in the context of Wnt polypeptides, a chaperone is an isolated or exogenous protein or fragments thereof, that is added in vitro to a solution comprising isolated Wnt polypeptides. In some cases, the isolated Wnt polypeptides are Wnt polypeptides that have been harvested and purified from a cell solution.

In some embodiments, a chaperone comprises Frizzled-8. Frizzled-8, encoded by the FZD8 gene, is a seven-transmembrane domain protein and a receptor for Wnt polypeptides.

In some instances, human Frizzled-8 (NCBI Reference Seq: NP_114072.1; SEQ ID NO: 4) comprises 694 amino acids in length. In some cases, Frizzled-8 comprises a 27 amino acid signal sequence, a 248 amino acid extracellular N-terminus, and an 89 amino acid C-terminus. In some cases, the N-terminus further comprises two putative N-linked glycosylation sites, a polyproline segment and a polyglycine segment. In addition, the N-terminus comprises a cysteine-rich domain (CRD) that is about 120 amino acids in length. The C-terminus of Frizzled-8 comprises a Thr-x-Val tripeptide, a Lys-Thr-x-x-x-Trp motif, and a polyglycine repeat of 25 amino acids in length.

In some instances, a Frizzled-8 polypeptide described herein comprises about 70%, 75%, 80%, 85%, 90%, 95%, 96%, 97%, 98%, or 99% sequence identity to human Frizzled-8. In some cases, a Frizzled-8 polypeptide described herein comprises about 70%, 75%, 80%, 85%, 90%, 95%, 96%, 97%, 98%, or 99% sequence identity to SEQ ID NO: 4.

In some embodiments, a chaperone described herein comprises a Frizzled-8 fusion protein. In some cases, the Frizzled-8 fusion protein comprises a truncated Frizzled-8 protein. In some instances, the truncated Frizzled-8 protein comprises a cysteine-rich region (CRD) of Frizzled-8. In some instances, the truncated Frizzled-8 protein comprises the region spanning amino acid residue 25 to amino acid residue 172 of SEQ ID NO: 4.

In some instances, the Frizzled-8 fusion protein further comprises the Fc portion of an antibody. In some instances, the antibody is selected from IgA, IgD, IgE, IgG or IgM. In some cases, the antibody is IgG. In some cases, the Frizzled-8 fusion protein comprises a truncated Frizzled-8 protein (e.g., the CRD portion of Frizzled-8) and an IgG Fc portion.

In some cases, the truncated Frizzled-8 protein is covalently linked to the Fc portion directly. In other cases, the truncated Frizzled-8 protein is covalently linked to the Fc portion indirectly via a linker. In some instances, a linker comprises a series of glycines, alanines, or a combination thereof. In some instances, a linker comprises the amino acid sequence IEGRMD (SEQ ID NO: 6).

In some cases, the Frizzled-8 fusion protein comprises at least 80%, 85%, 90%, 95%, 96%, 97%, 98%, or 99% sequence identity to SEQ ID NO: 5. In some cases, the Frizzled-8 fusion protein comprises at least 80% sequence identity to SEQ ID NO: 5. In some cases, the Frizzled-8 fusion protein comprises at least 85% sequence identity to SEQ ID NO: 5. In some cases, the Frizzled-8 fusion protein comprises at least 90% sequence identity to SEQ ID NO: 5. In some cases, the Frizzled-8 fusion protein comprises at least 95% sequence identity to SEQ ID NO: 5. In some cases, the Frizzled-8 fusion protein comprises at least 96% sequence identity to SEQ ID NO: 5. In some cases, the Frizzled-8 fusion protein comprises at least 97% sequence identity to SEQ ID NO: 5. In some cases, the Frizzled-8 fusion protein comprises at least 98% sequence identity to SEQ ID NO: 5. In some cases, the Frizzled-8 fusion protein comprises at least 99% sequence identity to SEQ ID NO: 5. In some cases, the Frizzled-8 fusion protein comprises 100% sequence identity to SEQ ID NO: 5. In some cases, the Frizzled-8 fusion protein consists the sequence set forth in SEQ ID NO: 5.

In some embodiments, a chaperone described herein comprises low-density lipoprotein receptor-related protein 5 (LRPS) or low-density lipoprotein receptor-related protein 6 (LRP6). LRPS and LRP6 are type I, single-pass transmembrane glycoproteins that act as co-receptors to the Wnt family of proteins. In some instances, LRPS comprises a 24 amino acid signal sequence, a 1361 amino acid extracellular region, a 23 amino acid TM domain and a 207 amino acid cytoplasmic tail. In some cases, LRP6 comprises a 19 aa signal sequence, a 1353 aa extracellular domain, a 23 aa TM segment, and a 218 aa cytoplasmic tail. In some embodiments, a chaperone described herein is LRP6.

In some embodiments, an isolated Wnt polypeptide and a plurality of chaperones are incubated for at least 10 minutes, at least 30 minutes, at least 1 hour, at least 1.5 hour, at least 2 hours, at least 3 hours, at least 4 hours, at least 5 hours, at least 6 hours, at least 10 hours, at least 12 hours, at least 18 hours, or more. In some instances, the isolated Wnt polypeptide and the plurality of chaperones are incubated for at least 10 minutes or more. In some instances, the isolated Wnt polypeptide and the plurality of chaperones are incubated for at least 30 minutes or more. In some instances, the isolated Wnt polypeptide and the plurality of chaperones are incubated for at least 1 hour or more. In some instances, the isolated Wnt polypeptide and the plurality of chaperones are incubated for at least 2 hours or more. In some instances, the isolated Wnt polypeptide and the plurality of chaperones are incubated for at least 3 hours or more. In some instances, the isolated Wnt polypeptide and the plurality of chaperones are incubated for at least 4 hours or more. In some instances, the isolated Wnt polypeptide and the plurality of chaperones are incubated for at least 5 hours or more. In some instances, the isolated Wnt polypeptide and the plurality of chaperones are incubated for at least 6 hours or more. In some instances, the isolated Wnt polypeptide and the plurality of chaperones are incubated for at least 10 hours or more. In some instances, the isolated Wnt polypeptide and the plurality of chaperones are incubated for at least 12 hours or more. In some instances, the isolated Wnt polypeptide and the plurality of chaperones are incubated for at least 18 hours or more. In some instances, the isolated Wnt polypeptide and the plurality of chaperones are incubated for at least 24 hours or more. In some cases, the isolated Wnt polypeptide is obtained from a minimal serum condition and in the absence of liposome. In other cases, the isolated Wnt polypeptide is formulated as a liposomal Wnt polypeptide prior to incubation with a chaperone for further purification.

In some embodiments, an isolated Wnt polypeptide and a plurality of chaperones are incubated at a temperature of between about 1° C. and about 30° C. In some cases, the isolated Wnt polypeptide and the plurality of chaperones are incubated at a temperature of between about 1° C. and about 10° C., between about 1° C. and about 8° C., or between about 1° C. and about 4° C. In some cases, the isolated Wnt polypeptide and the plurality of chaperones are incubated at a temperature of between about 10° C. and about 30° C., between about 15° C. and about 30° C., between about 20° C. and about 30° C., between about 23° C. and about 30° C., or between about 25° C. and about 30° C. In some cases, the isolated Wnt polypeptide and the plurality of chaperones are incubated at a temperature of between about 1° C. and about 10° C. In some cases, the isolated Wnt polypeptide and the plurality of chaperones are incubated at a temperature of between about 1° C. and about 8° C. In some cases, the isolated Wnt polypeptide and the plurality of chaperones are incubated at a temperature of between about 1° C. and about 4° C. In some cases, the isolated Wnt polypeptide and the plurality of chaperones are incubated at a temperature of between about 10° C. and about 30° C. In some cases, the isolated Wnt polypeptide and the plurality of chaperones are incubated at a temperature of between about 15° C. and about 30° C. In some cases, the isolated Wnt polypeptide and the plurality of chaperones are incubated at a temperature of between about 20° C. and about 30° C. In some cases, the isolated Wnt polypeptide and the plurality of chaperones are incubated at a temperature of between about 23° C. and about 30° C. In some cases, the isolated Wnt polypeptide and the plurality of chaperones are incubated at a temperature of between about 25° C. and about 30° C. In some cases, the isolated Wnt polypeptide are obtained from a minimal serum condition and in the absence of liposome. In other cases, the isolated Wnt polypeptide are formulated as liposomal Wnt polypeptides.

In some cases, an isolated Wnt polypeptide and the plurality of chaperones are incubated at a temperature of at least 1° C., 2° C., 4° C., 8° C., 10° C., 20° C., 23° C., 25° C., or 30° C. In some cases, the isolated Wnt polypeptide and the plurality of chaperones are incubated at a temperature of at least 1° C. In some cases, the isolated Wnt polypeptide and the plurality of chaperones are incubated at a temperature of at least 2° C. In some cases, the isolated Wnt polypeptide and the plurality of chaperones are incubated at a temperature of at least 4° C. In some cases, the isolated Wnt polypeptide and the plurality of chaperones are incubated at a temperature of at least 8° C. In some cases, the isolated Wnt polypeptide and the plurality of chaperones are incubated at a temperature of at least 10° C. In some cases, the isolated Wnt polypeptide and the plurality of chaperones are incubated at a temperature of at least 20° C. In some cases, the isolated Wnt polypeptide and the plurality of chaperones are incubated at a temperature of at least 23° C. In some cases, the isolated Wnt polypeptide and the plurality of chaperones are incubated at a temperature of at least 25° C. In some cases, the isolated Wnt polypeptide and the plurality of chaperones are incubated at a temperature of at least 30° C. In some cases, the isolated Wnt polypeptide are obtained from a minimal serum condition and in the absence of liposome. In other cases, the isolated Wnt polypeptide is formulated as a liposomal Wnt polypeptide prior to incubation with a chaperone for further purification.

In some embodiments, isolated Wnt polypeptides and a plurality of chaperones are incubated at a ratio of about 1:0.5, 1:1, 1:1.5, 1:2, 1:2.5, 1:3, 1:4, or about 1:5 Wnt polypeptide:chaperone. In some cases, the isolated Wnt polypeptides and the plurality of chaperones are incubated at a ratio of about 1:0.5 Wnt polypeptide:chaperone. In some cases, the isolated Wnt polypeptides and the plurality of chaperones are incubated at a ratio of about 1:1 Wnt polypeptide:chaperone. In some cases, the isolated Wnt polypeptides and the plurality of chaperones are incubated at a ratio of about 1:1.5 Wnt polypeptide:chaperone. In some cases, the isolated Wnt polypeptides and the plurality of chaperones are incubated at a ratio of about 1:2 Wnt polypeptide:chaperone. In some cases, the isolated Wnt polypeptides and the plurality of chaperones are incubated at a ratio of about 1:2.5 Wnt polypeptide:chaperone. In some cases, the isolated Wnt polypeptides and the plurality of chaperones are incubated at a ratio of about 1:3 Wnt polypeptide:chaperone. In some cases, the isolated Wnt polypeptides and the plurality of chaperones are incubated at a ratio of about 1:4 Wnt polypeptide:chaperone. In some cases, the isolated Wnt polypeptides and the plurality of chaperones are incubated at a ratio of about 1:5 Wnt polypeptide:chaperone. In some cases, the isolated Wnt polypeptides are obtained from a minimal serum condition and in the absence of liposome. In other cases, the isolated Wnt polypeptides are formulated as liposomal Wnt polypeptides prior to incubation with a chaperone for further purification.

In some embodiments, each of the plurality of chaperones is further immobilized on a bead. In some cases, each chaperone is immobilized directly on the bead. In other cases, each chaperone is immobilized indirectly on the bead.

In some embodiments, each of the plurality of chaperones comprises a Frizzled-8 fusion protein. In some cases, a Frizzled-8 fusion protein is directly immobilized on a bead. In other cases, a Frizzled-8 fusion protein is indirectly immobilized on a bead, in which the Frizzled-8 fusion protein is bound to a polypeptide that recognizes the Fc portion of an antibody, and wherein the polypeptide is immobilized to the bead. In some cases, the polypeptide is Protein A.

In some instances, a separation step is performed to elute a Wnt polypeptide-chaperone complex and/or an isolated Wnt polypeptide from the plurality of beads. In some cases, the separation step is carried out in batch mode. In other instances, the separation step is carried out using a column immobilized with a chaperone and/or a chaperone further bound to a polypeptide that recognizes the Fc portion of an antibody (e.g., Protein A). In some cases, a buffer comprising an acidic pH is used for the separation step (or the elution step). In some cases, the buffer comprises a pH of about 2, 2.5, 3. 3.5, 4, 5 or about 6. In some cases, the buffer comprises a pH of about 3.

In some cases, a step gradient is used to elute a Wnt polypeptide-chaperone complex and/or an isolated Wnt polypeptide from the plurality of beads. In some cases, the step gradient comprises a first gradient and a second gradient. In some cases, the first gradient comprises a first buffer comprising a salt concentration of at most 0, 0.01, 5, 10, 15, 20, 25, 30, 40, 50 mM, or less. In some cases, the first gradient comprises a first buffer comprising a salt concentration of at least 0, 0.01, 5, 10, 15, 20, 25, 30, 40, 50 mM, or more. In some cases, the first buffer comprising the first gradient is used as a wash step to remove unbound impurities (e.g., uncomplexed Wnt polypeptides and/or chaperones). In some embodiments, at most 1, 2, 3, 4, 5, or more wash steps are used. In some embodiments, at least 1, 2, 3, 4, 5 or less wash steps are used. In some embodiment, the second gradient comprises a second buffer comprising a salt concentration of at least 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000 mM, or more. In some embodiment, the second gradient comprises a second buffer comprising a salt concentration of at most 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000 mM, or less. Exemplary salt include sodium chloride, potassium chloride, magnesium chloride, calcium chloride, calcium phosphate, potassium phosphate, magnesium phosphate, sodium phosphate, ammonium sulfate, ammonium chloride, ammonium phosphate, and the like.

In some embodiments, a detergent is also formulated into the first and/or second buffer. In some embodiments, the detergent is CHAPS or Triton X-100. In some embodiments, the percentage of CHAPS or Triton X-100 is at least 0.01%, 0.1%, 0.5%, 1%, 1.5%, 2%, 2.5%, 3%, 3.5%, 4%, 4.5%, 5%, or more. In some embodiments, the percentage of CHAPS or Triton X-100 is at most 0.01%, 0.1%, 0.5%, 1%, 1.5%, 2%, 2.5%, 3%, 3.5%, 4%, 4.5%, 5%, or less. In some instances, buffer components such as tris(hydroxymethyl)methylamine HCl (Tris-HCl), 3-{[tris(hydroxymethyl)methyl]amino}propanesulfonic acid (TAPS), N,N-bis(2-hydroxyethyl)glycine (Bicine), N-tris(hydroxymethyl)methylglycine (Tricine), 3-[N-Tris(hydroxymethyl)methylamino]-2-hydroxypropanesulfonic acid (TAPSO), 4-2-hydroxyethyl-1-piperazineethanesulfonic acid (HEPES), 3-(N-morpholino)propanesulfonic acid (MOPS), piperazine-N,N′-bis(2-ethanesulfonic acid) (PIPES), 2-(N-morpholino)ethanesulfonic acid (MES), and the like, are used.

In some instances, the pH of the first and/or second buffer is at least 4, 4.5, 5, 5.5, 6, 6.5, 7, 7.5, 8, 8.5, 9, or more. In some instances, the pH of the first and/or second buffer is at most 4, 4.5, 5, 5.5, 6, 6.5, 7, 7.5, 8, 8.5, 9, or less.

In some instances, an additional elution step is used to elute an isolated Wnt polypeptide from a Wnt polypeptide-chaperone complex. In some instances, an elution buffer, for example, comprises a first gradient and a second gradient as described above and/or comprising a detergent is used to elute the isolated Wnt polypeptide from the Wnt polypeptide-chaperone complex.

In some instances, an aqueous solution of liposome is used to elute an isolated Wnt polypeptide from a Wnt polypeptide-chaperone complex, to generate a liposomal Wnt polypeptide. In some instances, the chaperone is a Frizzled-8 fusion protein. In some cases, an aqueous solution of liposome is used to elute the isolated Wnt polypeptide from the Wnt polypeptide-Frizzled-8 complex.

### Wnt Polypeptide Composition

Compositions are provided where the biologically active Wnt polypeptide secreted into serum-free medium is provided in a serum-free medium or a pharmaceutically acceptable excipient at a concentration of at least about 0.1 μg/ml; at least about 0.25 μg/ml; at least about 0.5 μg/ml; at least about 0.75 μg/ml; at least about 1 μg/ml; at least about 2.5 μg/ml; at least about 5 μg/ml; at least about 7.5 μg/ml; at least about 10 μg/ml; at least about 25 μg/ml; at least about 50 μg/ml; at least about 75 μg/ml; at least about 100 μg/ml; at least about 250 μg/ml; at least about 500 μg/ml; at least about 750 μg/ml; at least about 1 mg/ml; at least about 2.5 mg/ml; at least about 5 mg/ml; at least about 7.5 mg/ml; at least about 10 mg/ml; at least about 25 mg/ml; at least about 50 mg/ml; at least about 75 mg/ml; at least about 100 mg/ml; or more.

In some embodiments, the Wnt polypeptide produced by the methods and culture systems of the invention is purified by subjecting the medium to purification on a Blue Sepharose ion-exchange column in the absence of a gel filtration purification step. In one embodiment, the purification is performed also in the absence of a purification step of a heparin sulfate column. In a further embodiment, purification on the Blue Sepharose ion-exchange column is performed using a salt gradient of 150 mM to 1.0 M, where the salt may, for example be sodium or potassium chloride. In other embodiments, the Wnt polypeptide is purified by complexing an isolated Wnt polypeptide with a chaperone, and elution of the isolated Wnt polypeptide from the Wnt-chaperone complex. The purification scheme may be followed by formulation into liposomes. For various purposes, such as stable storage, the protein may be lyophilized. Lyophilization is preferably performed on an initially purified preparation, e.g. of at least about 1 mg/ml. Components may be added to improve the protein stability, e.g. lipids, detergents, etc.

The protein produced by the methods and culture systems of the invention can be incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, or liquid forms, such as tablets, capsules, powders, granules, ointments, solutions, suppositories, injections, inhalants, gels, microspheres, etc. As such, administration of the protein and/or other compounds can be achieved in various ways. The protein and/or other compounds may be systemic after administration or may be localized by virtue of the formulation, or by the use of an implant that acts to retain the active dose at the site of implantation.

In pharmaceutical dosage forms, the protein and/or other compounds may be administered in the form of their pharmaceutically acceptable salts, or they may also be used alone or in appropriate association, as well as in combination with other pharmaceutically active compounds. The agents may be combined to provide a cocktail of activities. The following methods and excipients are exemplary and are not to be construed as limiting the invention.

Pharmaceutical formulations may be provided in a unit dosage form, where the term “unit dosage form,” refers to physically discrete units suitable as unitary dosages for human subjects, each unit containing a predetermined quantity of protein in an amount calculated sufficient to produce the desired effect in association with a pharmaceutically acceptable diluent, carrier or vehicle. The specifications for the unit dosage forms of the present invention depend on the particular composition employed and the effect to be achieved, and the pharmacodynamics associated with the composition in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Depending on the patient and condition being treated and on the administration route, the protein may be administered in dosages of 0.001 mg to 500 mg/kg body weight per day, e.g. about 0.1-100 mg/kg body weight/per day, e.g., 20 mg/kg body weight/day for an average person.

Those of skill will readily appreciate that dose levels can vary as a function of the specific enzyme, the severity of the symptoms and the susceptibility of the subject to side effects. Some of the proteins are more potent than others. Preferred dosages for a given enzyme are readily determinable by those of skill in the art by a variety of means. A preferred means is to measure the physiological potency of a given compound.

The compositions of the invention can be used for prophylactic as well as therapeutic purposes. As used herein, the term “treating” refers both to the prevention of disease and the treatment of a disease or a pre-existing condition and more generally refers to the enhancement of Wnt3A activity at a desired tissue, site, timing, etc. The invention provides a significant advance in the treatment of ongoing disease, and helps to stabilize and/or improve the clinical symptoms of the patient. Such treatment is desirably performed prior to loss of function in the affected tissues but can also help to restore lost function or prevent further loss of function. Evidence of therapeutic effect may be any diminution in the severity of disease or improvement in a condition, e.g. enhanced bone healing, etc. The therapeutic effect can be measured in terms of clinical outcome or can be determined by biochemical tests. Alternatively, one can look for a reduction in symptoms of a disease.

In other embodiments of the invention, cell compositions are provided, where the cells comprise an expression vector comprising a C-terminal truncated Wnt3A protein comprising a signal sequence for secretion, which can be the native Wnt3A signal sequence or a heterologous signal sequence, operably linked to a promoter. In some embodiments the cells are CHO-S cells. In some embodiments the cells are provided as a composition comprising serum-free culture medium. In other embodiments the cells are frozen and viable, and are optionally provided in aliquots suitable for seeding cultures.

Cells may be provided in a container, e.g. frozen aliquots, at concentrations of from about 10³ cells/ml, 10⁴ cells/ml, 10⁵ cells/ml, 10⁶ cells/ml, 10⁷ cells/ml, up to about 10⁸ cells/ml or more. Cells can be frozen in any suitable medium to maintains the viability of the cells, and may include DMSO. Cell compositions can be provided in a GMP format for example compositions useful in a master cell bank or working cell bank, which are derived from a single host cell under defined conditions and cloning history, then dispensed into multiple containers.

In some embodiments, the specific activity of a Wnt protein in a composition is measured by determining the level of activity in a functional assay, e.g. stabilization of 3-catenin, promoting growth of stem cells, etc., quantitating the amount of Wnt protein present in a non-functional assay, e.g. immunostaining, ELISA, quantitation on coomasie or silver stained gel, etc., and determining the ratio of biologically active Wnt to total Wnt. Generally, the specific activity as thus defined in a substantially homogeneous composition will be at least about 5% that of the starting material, usually at least about 10% that of the starting material, and may be about 25%, about 50%, about 90% or greater.

Assays for biological activity of Wnt include stabilization of 3-catenin, which can be measured, for example, by serial dilutions of the Wnt composition. An exemplary assay for Wnt biological activity contacts a Wnt composition with cells, e.g. mouse L cells. The cells are cultured for a period of time sufficient to stabilize 3-catenin, usually at least about 1 hour, and lysed. The cell lysate is resolved by SDS PAGE, then transferred to nitrocellulose and probed with antibodies specific for β-catenin. Other assays include C57MG transformation and induction of target genes in Xenopus animal cap assays.

### Kits/Article of Manufacture

Disclosed herein, in certain embodiments, are kits and articles of manufacture for use with one or more methods, processes, and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some embodiments, the containers are formed from a variety of materials such as glass or plastic.

The articles of manufacture provided herein contain packaging materials. Examples of packaging materials include, but are not limited to, bottles, tubes, bags, containers, bottles, and any packaging material suitable for a selected formulation and intended mode of administration and treatment.

For example, the container(s) include Wnt proteins. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

Further details of the invention are provided in the following non-limiting Examples.

### Example 1

### Production and Secretion into Serum Free Medium of Human Wnt3A Polypeptide

WNT3A is a lipid-modified human stem cell growth factor that is effective in activating adult stem cells and stimulating their self-renewal and survival. The protein is post-translationally modified by glycosylation and palmitoylation.

Two general methods were simultaneously employed to develop a serum free process; first, the gradual adaptation of a serum-expressing cell line to serum free conditions was attempted. Second, a new cell line was developed. For the first approach, a minimum of 5 separate serum substitutes was tested in an effort to replace the role of serum in WNT protein secretion. These substitutes included commercially available Excyte®, Cell-Ess, lipid mix supplements, and ITS (Insulin-Transferrin-Selenium) supplements. For the second approach, all of the following combinations were rigorously evaluated: 1. GMP compatible cell lines for WNT3A production were identified, that included CHO-K, CHO-S, DG44, and TReX 2. Both cDNA clones encoding WNT3A were tested (e.g., BC103922 and BC103921). GMP compatible vectors for cloning were identified, that included OpticVec™ (expression vector), pTarget™ (expression vector), and pcDNA™4TO4 (expression vector). Two methods were used for transfection (stable and transient) 5. Two methods were tested for induction (doxycycline and tetracycline). All of these methods resulted in the strong expression, but not secretion, of WNT3A from CHO cell lines. In some cases, very small amounts of WNT3A was found in the conditioned media but in no cases did this protein exhibit function.

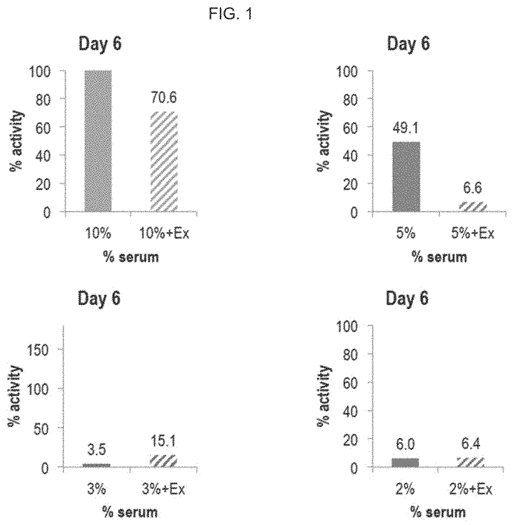

The first approach was further illustrated by FIGS. 1-3. FIG. 1 illustrates Wnt3A activity in the presence of serum substitute Excyte® and decreasing serum concentrations. Wnt polypeptide is from an expression vector encoding the protein sequence set forth in SEQ ID NO:1. Wnt3A activity in conditioned media from cells adapted to 5% serum+Excyte® (blue dashed bar), 3% serum+Excyte® (red dashed bar) and 2% serum+Excyte® (purple dashed bar) was analyzed using dual light reporter assay. This activity was compared to activity of conditioned media from cells adapted to 5% serum (blue solid bar), 3% serum (red solid bar) and 2% serum (purple solid bar) without Excyte® supplement. The condition media from cells grown in 10% serum (orange bar) was used as a positive control. As compared to 10% FBS the activity of conditioned media from cells adapted to 2% serum and 2% serum+Excyte® was reduced to 6.4%. Decreasing serum concentrations resulted in reduced Wnt3A activity in the conditioned media. Addition of Excyte® did not have an effect on Wnt3A activity in conditioned media.

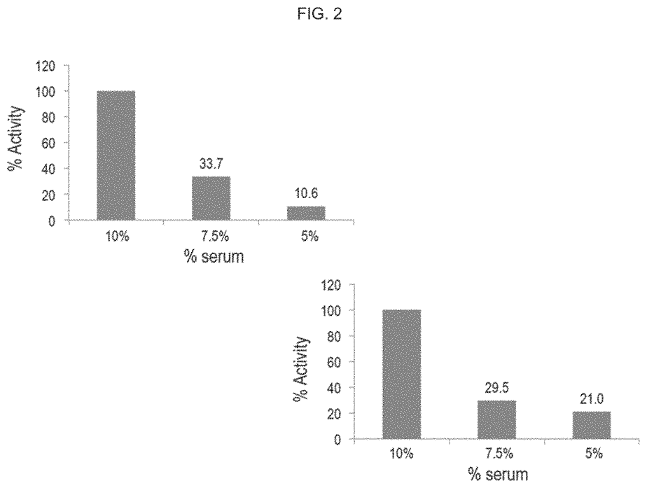

FIG. 2 shows Wnt3A activity in the presence of serum substitute CellEss® and decreasing serum concentrations. The Wnt3A polypeptide is from an expression vector encoding the protein sequence set forth in SEQ ID NO:1. Wnt3A activity in conditioned media from cells adapted to 7.5% and 5% serum supplemented with Excyte® was analyzed using a dual light reporter assay. This activity was compared to Wnt3A activity in condition media from cells grown in 10% serum. Presence of CellEsse in the culture media was not able to restore Wnt3A activity in the conditioned media.

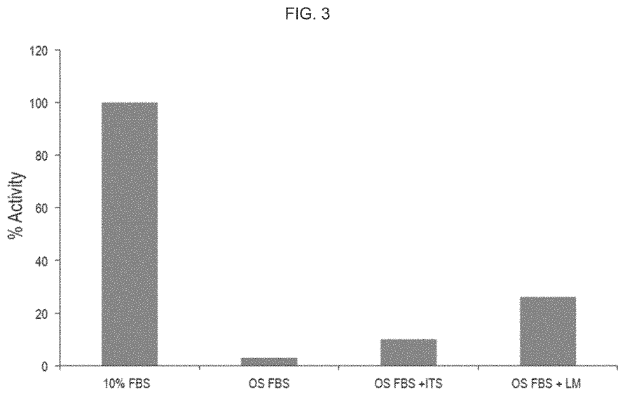

FIG. 3 shows Wnt3A activity from an expression vector encoding the protein sequence set forth in SEQ ID NO:1. Cells were first adapted to charcoal stripped one shot FBS (OS FBS). No detectable activity was measured in conditioned media from cells adapted to OSFBS. Following adaptation to OSFBS, OSFBS was supplemented with either ITS3 (Insulin-Transferrin-Selenium-3) or lipid mix 1. WNT3A activity in conditioned media was tested using the LSL dual light reporter assay. Conditioned media from cells adapted to OSFBS+ITS (Insulin-Transferrin-Selenium) sample demonstrated ˜10% of activity when compared to the positive control (10% FBS). Conditioned media from cells adapted to OSFBS+lipid mix sample demonstrated 26% of activity when compared to the positive control, 10% FBS.

In the second approach, culture conditions were developed that allow for the efficient secretion of Wnt3A in the absence of serum. A CHO-K1 derivative cell line (e.g., CHO-S) was identified that efficiently secretes Wnt3A under serum free conditions. CHO-S cells were transiently transfected with a pcDNA™4.0 vector containing the WNT3A cDNA (BC103922 encoding the Homo sapiens wingless-type MMTV integration site family, member 3A, mRNA complete coding sequence). Conditioned media (CM) harvested from the cells was applied to WNT reporter (LSL) cells; CHO-S cells transfected with a GFP expression plasmid served as a control. This activity assay along with a Western blot analysis of the CM demonstrates Wnt3A secretion in the absence of serum or any other animal component.

Conditioned media (CM) from CHO cell cultures were collected between day 3 and day 13 after induction and pooled. Based on analyses of protein production in the CM on each day, this range of days was determined to be optimal under the culture conditions used in the present Example. Under these conditions, highest protein production occurred between days 3-13, while after day 13 cells began to die.

### Example 2

### Mouse LSL Cell-Based Assay

Mouse LSL cells are stably transfected with a Wnt-responsive luciferase reporter plasmid pSuperTOPFlash (Addgene) and a constitutive LacZ expression construct pEF/Myc/His/LacZ (Invitrogen) for normalizing beta galactosidase activity to cell number. Human embryonic kidney epithelial (HEK293T) cells are stably transfected with the above two plasmids. Cells (50000 cells/well, 96-well plate) are treated with L-WNT3A in DMEM supplemented with 10% FBS (Gibco) and 1% P/S (Cellgro) at a concentration of 10 uL in 150 uL total volume, unless otherwise stated. Included also was a serial dilution of purified WNT3A protein.

Cells are incubated overnight at 37° C., 5% CO₂, then washed, lysed with Lysis Buffer (Applied Biosystems), and the luciferase and 3-galactosidase expression levels quantified using a dual-light combined reporter gene assay system (Applied Biosysytems). Bioluminescence was quantified with triplicate reads on a dual light ready luminometer (Berthold). Activity of WNT3A (ng/uL) and L-WNT3A is defined from a standard curve generated by serial dilutions of WNT3A protein. In experiments involving a time course, WNT3A activity is expressed as percent activity. Percent activity is calculated as follows:

$$\% activity = \frac{( \frac{luc}{lac} ) tn}{( \frac{luc}{lac} ) t 0} * 100$$

L-WNT3A dose response curves using primary MEFs. LSL and HEK293T cells are engineered to be maximally sensitive to Wnt and Wnt agonists and therefore may not provide meaningful data on the relationship between dose, drug effect, and clinical response. To more closely mimic the in vivo cellular response to a Wnt stimulus, mouse embryonic fibroblasts (MEFs) using expression of the Wnt target gene Axin2 as a measure of pathway activity are assayed.

### Example 3

### Lipid Reconstitution of WNT3A

Many proteins denature at high temperatures, and avoiding such denaturation at body temperature is key to extending the duration of a protein therapeutic. Liposomal packaging preserves the biological activity of Wnt3A and that this formulation has efficacy in multiple bone injury applications. After purification, recombinant Wnt3A is reconstituted into lipid vesicles consisting of DMPC and cholesterol.

In some aspects, a L-WNT3A formulation is to be used in an investigational new drug (IND) Phase I study to treat bone defects in patients at high risk for delayed bone healing. In some instances, autologous bone graft material (BGM) is harvested, treated with L-WNT3A ex vivo, then washed and pelleted. In some cases, the resulting material, activated BGM (e.g., BGM^{ACT}), is considered the drug product and is ready for immediate use. In some instances, L-WNT3A will not be directly administered to the patient but only used to activate the autologous cells ex vivo. In some cases, for the initial stages of the program it is expected that the formulation will meet accepted criteria (purity, stability, etc.) for a systemically administered liposomal protein formulation.

### Example 4

### Scale Up Experiment

Cells from the freezer stock are seeded onto a 15 cm tissue culture plate. After incubation at 37° C., 5% CO₂ for 3-4 days, the cells are expanded 1:5 into 2×15 cm plates for 4 days. These cells are further expanded 1:5 into 20×15 cm plates. After 24 hours of incubation the cells are induced with doxycycline. CM is collected every 24 hours and stored at 4° C. Activity of the CM is measured to confirm WNT3A secretion. 1% TritonX is added to 1 L CM and filtered through a 0.22 μm filter. CM is then loaded onto a 150 ml blue sepharose column. From this trial 80 μg of WNT3A is eluted in a gradient of KCl.

### Example 5

### Production and Secretion of Wnt3A Polypeptide in a CHO Cell Line

A CHO-K1 derivative (e.g., CHO-S) cell line was developed that secretes Wnt3A under serum free conditions. CHO-S cells were transiently transfected with a pcDNA™4.0 vector containing the WNT3A cDNA BC1 03922. Conditioned media (CM) was harvested after 2 days. To detect WNT3A activity WNT reporter cells (LSL) were treated with CM; CHO-S cells transfected with a GFP expression plasmid served as a control.

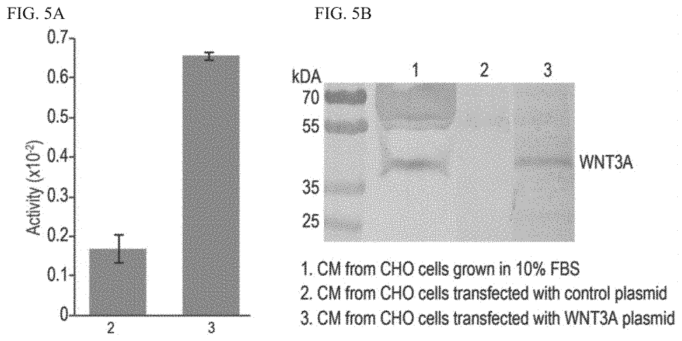

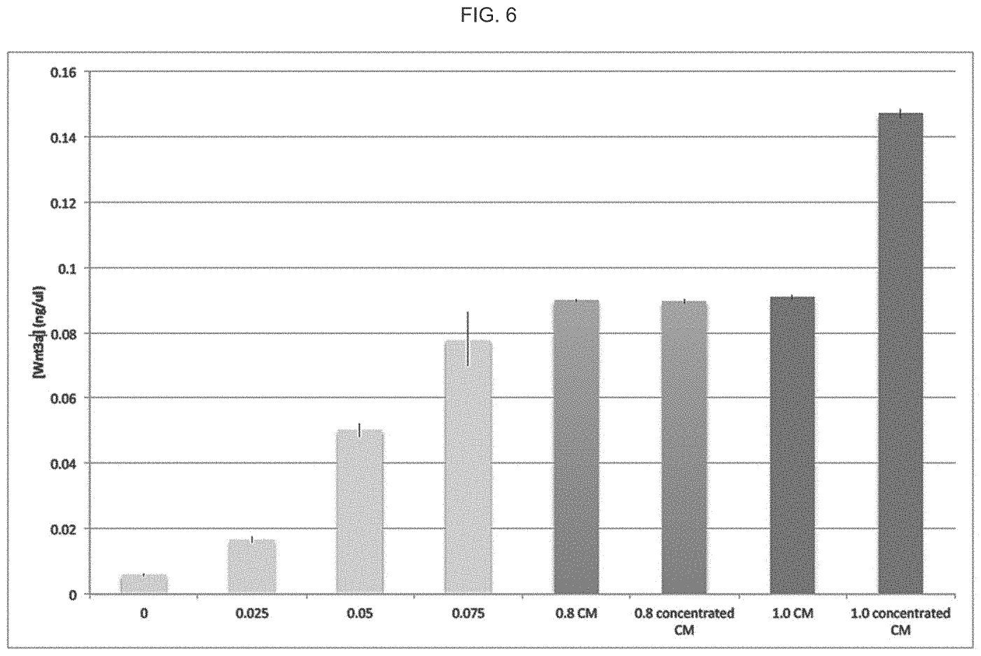

The activity assay illustrated in FIG. 5 shows that CM from CHO cells transfected with the GFP plasmid control exhibit baseline activity in the LSL reporter assay (FIG. 5A, lane 2 and 5B, lane 2). CM from CHO cells transfected with the BC103922 cDNA exhibit elevated activity in the LSL assay and W Western blot analysis confirms the presence of a band that runs at the same molecular weight as WNT3A (FIG. 5B, lane 1 and lane 3). Additional characterization has been carried out. Cells were selected with either 0.8 mg/mL or 1.0 mg/mL zeocin. The resulting cells were grown in serum free conditions. CM was collected and concentrated. Activity was measured using the LSL assay and compared to activity from purified WNT3A (FIG. 6, light blue bars). Activity was not detected in the clone under 0.8 mg/mL zeocin selection, even when the CM was concentrated (FIG. 6, medium blue bars). Activity was detected in a clone that was isolated using 1.0 mg/ml zeocin selection (FIG. 6, dark blue bars).

### Example 6

### Purification of Wnt3A Polypeptide with Frizzled-8 Fusion Protein

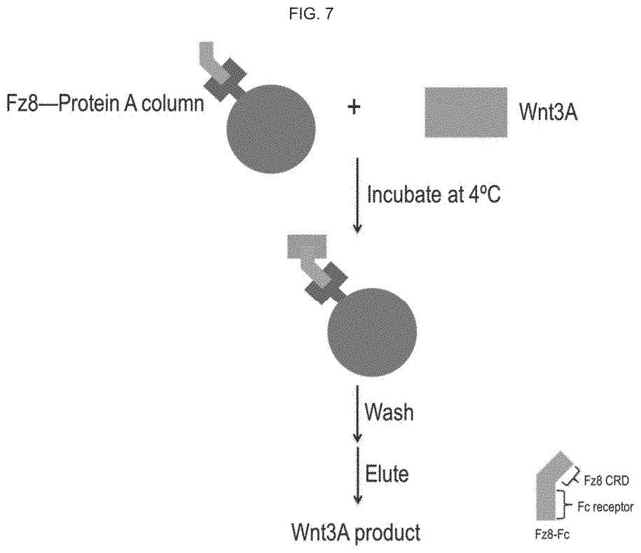

A Frizzled-8 fusion protein-Protein A purification scheme was utilized for purification of Wnt3A (FIG. 7). First, resin comprising Protein A immobilized beads was aliquoted at 50 μL and 25 μL volumes into two Eppendorf tubes. The resin in each tube was further washed with 20 column volumes of PBS. About 10 μL of Frizzled-8 fusion protein was added to each tube, with a final concentration of about 50 μg Frizzled-8/1 mL protein A or 100 μg Frizzled-8/1 mL protein A, respectively. The Frizzled-8 fusion protein was incubated for about 1.5-2 hours at 4° C. Post incubation, unbound Frizzled-8 fusion protein was removed with PBS.

Next, about 100 ng of Wnt3A in a PBS buffer with 1% CHAPS was incubated in one of the two tubes comprising Frizzled-8 fusion protein-Protein A resin for about 1.5-2 hours at 4° C. After incubation, unbound Wnt3A was removed with a PBS buffer comprising 1% CHAPS to remove unbound Wnt3A. The second tube was used as a control.

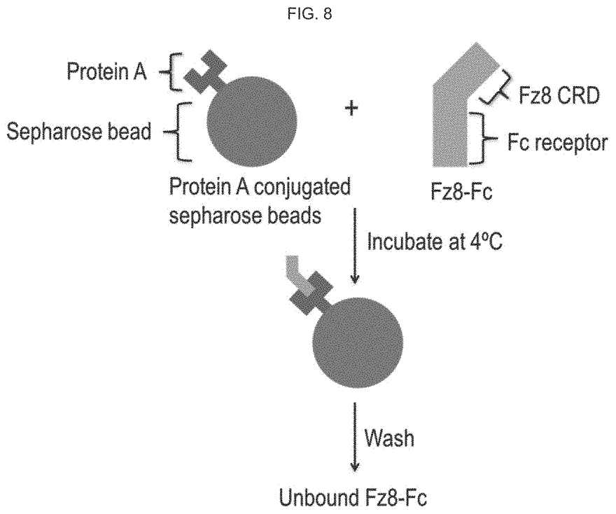

FIG. 8 illustrates a schematic showing a pre-complexation of a Frizzled-8 fusion protein with Protein A immobolized beads.

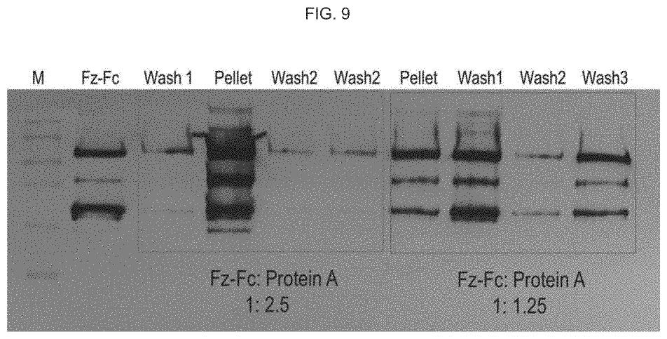

FIG. 9 illustrates a western blot showing complexation of Frizzled-8-Fc to Protein A at two different ratios.

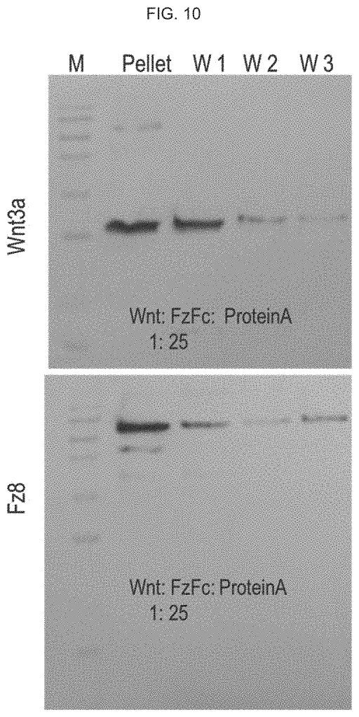

FIG. 10 illustrates a western blot showing Wnt3A purified using the Frizzled-8 fusion protein-Protein A strategy.

### Example 7

### Frizzled 8 and Liposomes Share the Same Binding Site on Wnt3A

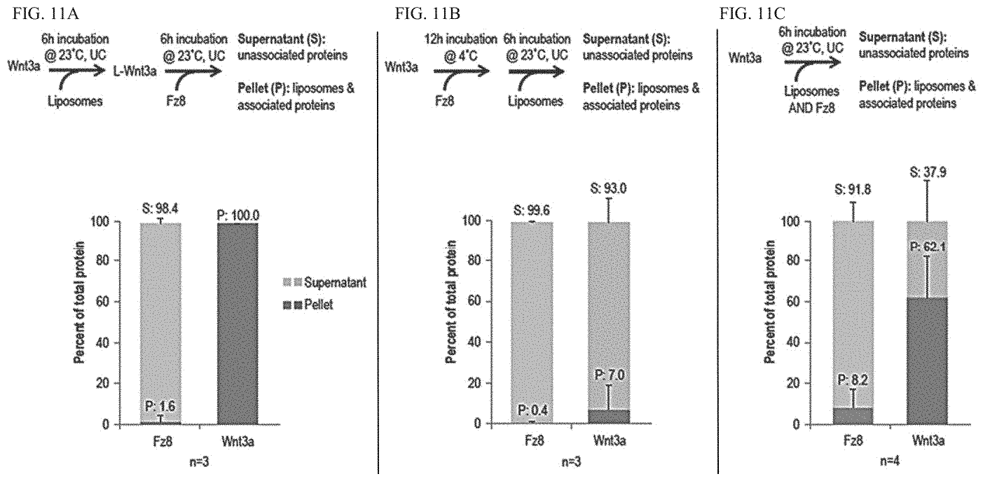

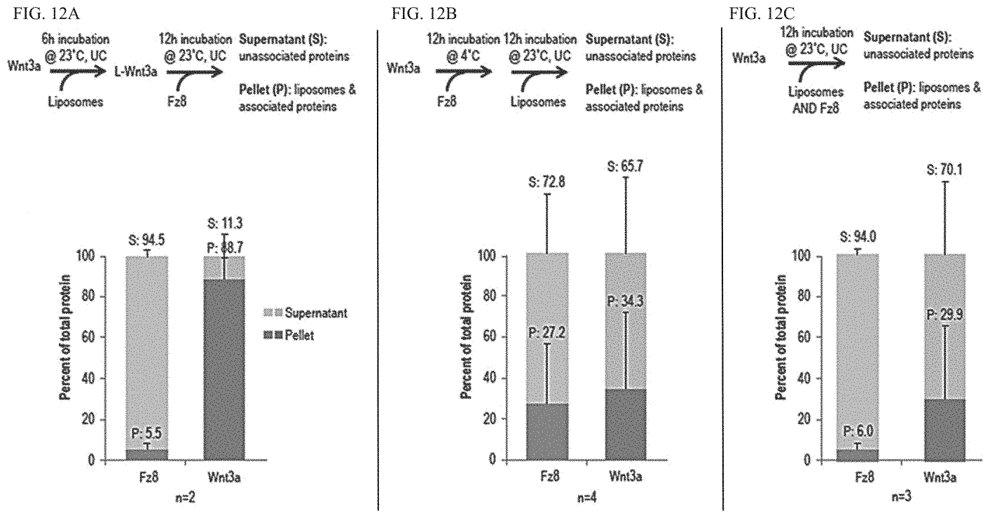

The crystal structure of Xenopus Wnt8 (XWnt8) in complex with mouse Frizzled 8 cysteine rich domain (Fz8-CRD) demonstrates that the Wnt8 lipid modification engages a groove on the Fz8-CRD, contacting nine Fz8 residues and traversing the cleft on the Fz8-CRD (PMID: 22653731). Based on the crystal structure of Xenopus Wnt8, it was hypothesized that liposomes maintain Wnt3A in an active conformation by directly interacting with the Wnt lipid modification, and the liposomal bilayer sterically shielding the lipid modification from the hydrophilic environment. To test this hypothesis, Wnt3A was first reconstituted into liposomes (L-Wnt3A) and then Fz8 was added to the L-Wnt3A solution. The samples were ultracentrifuged to separate the liposome associated proteins and unassociated proteins. Western blot analysis using Fz8 and Wnt3A antibodies demonstrated that ˜98% of the Fz8 was present in the supernatant, not associated with the liposomal pellet (light gray bar, FIG. 11A) and 100% of the Wnt3A was associated with the liposomal pellet (dark gray bar, FIG. 11A). To test if these interaction dynamics changed over time, L-Wnt3A was incubated with Fz8 for 12 h at room temperature (RT). In these conditions 94.5% of the Fz8 was present in the supernatant (light gray Fz8 bar, FIG. 12A), and 11% Wnt3A was present in the Fz8 rich supernatant (light gray Wnt3A bar FIG. 12A) while ˜89% Wnt3A was observed in the liposomal pellet (dark gray Wnt3A bar, FIG. 12A). These results showed a competition between Fz8 and liposomes for binding to Wnt3A, indicating that the Fz8 binding domain on Wnt3A is occluded by the liposomes and that Wnt3A separates based on its affinities.

To further test this hypothesis, Fz8 was first incubated with Wnt3A to facilitate a Fz8-Wnt3A interaction. After 12 h incubation at 4° C., liposomes were added and the sample was further incubated at 23° C. for 6 hours. These samples were ultracentrifuged to separate liposome-associated proteins from unassociated proteins. As observed in FIG. 11A, western blot analysis showed that >99% of the Fz8 protein was present in the supernatant (light gray bar, FIG. 11B). However, in these incubation conditions the distribution of Wnt3A changed: 93% of Wnt3A was present in the supernatant and only 7% of Wnt3A was associated with the liposomal pellet. These results showed that Fz8 and liposomes compete for binding to the same domain on Wnt3A.

Next, Wnt3A, liposomes and Fz8 were incubated for 6 h at RT. Following incubation of Wnt3A and liposomes for 6 h at RT, ˜90% of Wnt activity and Wnt protein were associated with the liposomal pellet (PMID: 24400074). Under these incubation conditions about 92% of the Fz8 was present in the supernatant (FIG. 11C, light gray bar) and 8% of Fz8 was present in the pellet (FIG. 11C, dark gray bar). About 62% of Wnt3A was present in the pellet (FIG. 11C, dark gray bar), as opposed to about 90% when only Wnt3A and liposomes were incubated together (PMID: 24400074). About 38% of Wnt3A was present in the supernatant fraction with Fz8 but if incubated for 12 h 70% of Wnt3A was observed in the supernatant (light gray Wnt3A bar, FIG. 12C). Wnt3A was either fractionated into liposomes or was present with Fz8 in the supernatant.

### Example 8

### Lrp6 Binding Site on Wnt3A is not Occluded by the Liposomes

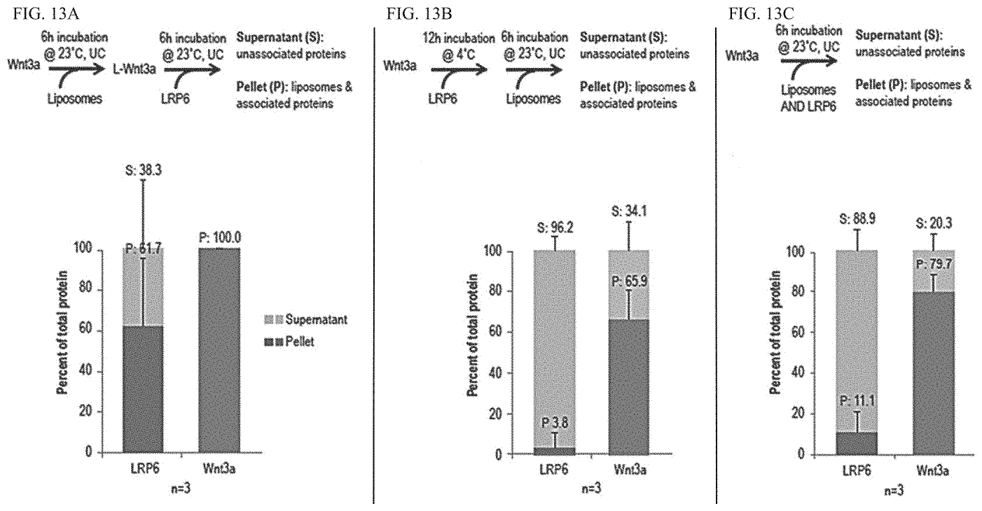

L-Wnt3A was incubated with Lrp6 at RT for 6 h. The samples were ultracentrifuged to remove liposome-unassociated proteins in the supernatant from the liposome-associated fraction in the pellet. About 62% of the Lrp6 was observed associated with the liposomes in the pellet (FIG. 13A). About 38% was observed in the supernatant (FIG. 13A). About 100% of L-Wnt3A was observed in the pellet (FIG. 13A). The majority of Lrp6 was found in the pellet along with Wnt3A and liposomes, suggesting that Lrp6 binds to a site not occluded by liposomes. Next, Wnt3A was pre-incubated with Lrp6 at 4° C. for 12 h to facilitate a Lrp6-Wnt3A interaction. This protein complex was further incubated with liposomes for 6 hours at room temperature and then ultracentrifuged to separate the liposome-associated fraction from the unassociated fraction. >96% of Lrp6 was present in the supernatant (FIG. 13B) and only about 3.8% Lrp6 was present in association with the liposomal pellet (FIG. 13B). Under these incubation conditions, about 34% Wnt3A was present in Lrp6 rich supernatant fraction (FIG. 13B). About 66% Wnt3A was present in liposomal pellet (FIG. 13B) as opposed to 100% as observed in FIG. 13A.

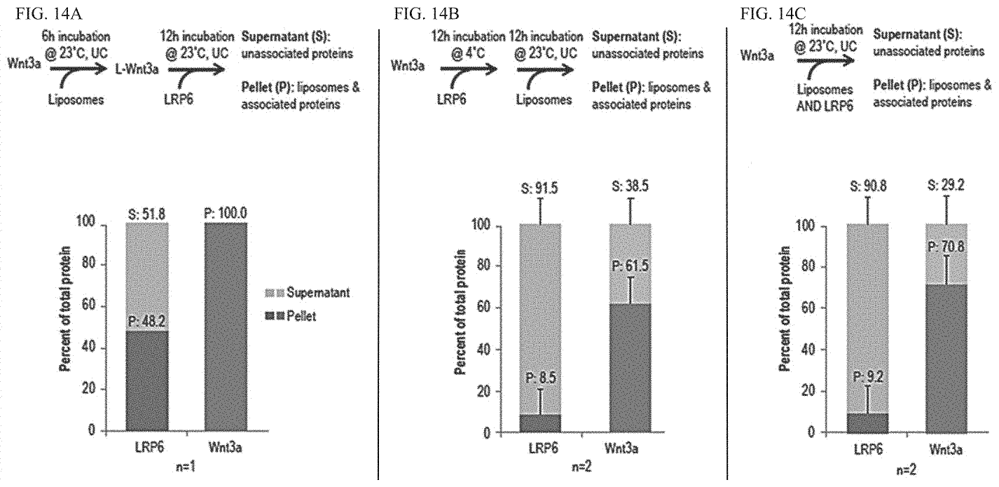

It was hypothesized that Wnt3A separates based on its affinity for liposomes and Lrp6. To test this Wnt3A, Lrp6 and liposomes were incubated together for six hours at 23° C. Western blot analysis of the supernatant and pellet showed that about 90% of Lrp6 was present in the supernatant (FIG. 13C) and about 11% was present in the liposomal pellet (FIG. 13C). About 20% Wnt3A was present in the supernatant (FIG. 13C). In these conditions more Wnt3A (70.8% vs. 61.5%) was associated with the liposomal pellet (FIG. 13C) when compared to conditions in FIG. 13B, indicating that Wnt3A has a lower binding affinity to LRP6 than to liposomes. In contrast to results of experiments involving Fz8 incubation (FIG. 11C, FIG. 12C), incubating for longer time period (12 hour) did not affect the Lrp6 and Wnt3A distribution (FIG. 14C). These experiments demonstrated that Lrp6 binds to a site on Wnt3A that is in a region exposed to the solvent and that Wnt3A has a lower binding affinity to LRP6 than to liposomes.

### Example 9

The following table illustrates Frizzled-8 and Frizzled-8 fusion protein sequences disclosed in this application.

| Protein Name | | SEQ ID NO: |
|---|---|---|
| Frizzled-8 | MEWGYLLEVTSLLAALALLQRSSGAAAASAKELACQEITVPLC | 4 |
| | KGIGYNYTYMPNQFNHDTQDEAGLEVHQFWPLVEIQCSPDLK | |
| | FFLCSMYTPICLEDYKKPLPPCRSVCERAKAGCAPLMRQYGF | |
| | AWPDRMRCDRLPEQGNPDTLCMDYNRTDLTTAAPSPPRRLP | |
| | PPPPGEQPPSGSGHGRPPGARPPHRGGGRGGGGGDAAAP | |
| | PARGGGGGGKARPPGGGAAPCEPGCQCRAPMVSVSSERH | |
| | PLYNRVKTGQIANCALPCHNPFFSQDERAFTVFWIGLWSVLC | |
| | FVSTFATVSTFLIDMERFKYPERPIIFLSACYLFVSVGYLVRLVA | |
| | GHEKVACSGGAPGAGGAGGAGGAAAGAGAAGAGAGGPGG | |
| | RGEYEELGAVEQHVRYETTGPALCTVVFLLVYFFGMASSIWW | |
| | VILSLTWFLAAGMKWGNEAIAGYSQYFHLAAWLVPSVKSIAVL | |
| | ALSSVDGDPVAGICYVGNQSLDNLRGFVLAPLVIYLFIGTMFLL | |
| | AGFVSLFRIRSVIKQQDGPTKTHKLEKLMIRLGLFTVLYTVPAA | |
| | VVVACLFYEQHNRPRWEATHNCPCLRDLQPDQARRPDYAVF | |
| | MLKYFMCLVVGITSGVWVWSGKTLESWRSLCTRCCWASKG | |
| | AAVGGGAGATAAGGGGGPGGGGGGGPGGGGGPGGGGGS | |
| | LYSDVSTGLTWRSGTASSVSYPKQMPLSQV | |
| Frizzled-8 | MEWGYLLEVTSLLAALALLQRSSGAAAASAKELACQEITVPLC | 5 |
| fusion protein | KGIGYNYTYMPNQFNHDTQDEAGLEVHQFWPLVEIQCSPDLK | |
| | FFLCSMYTPICLEDYKKPLPPCRSVCERAKAGCAPLMRQYGF | |
| | AWPDRMRCDRLPEQGNPDTLCMDYGGGGGGGDKTHTCPP | |
| | CPAPELLGGPSVFLFPPKPKDTLMISRTPEVTCVVVDVSHEDP | |
| | EVKFNWYVDGVEVHNAKTKPREEQYNSTYRVVSVLTVLHQD | |
| | WLNGKEYKCKVSNKALPAPIEKTISKAKGQPREPQVYTLPPSR | |
| | DELTKNQVSLTCLVKGFYPSDIAVEWESNGQPENNYKTTPPV | |
| | LDSDGSFFLYSKLTVDKSRWQQGNVFSCSVMHEALHNHYTQ | |
| | KSLSLSPGK | |

Although in the foregoing description the invention is illustrated with reference to certain embodiments, it is not so limited. Indeed, various modifications of the invention in addition to those shown and described herein will become apparent to those skilled in the art from the foregoing description and fall within the scope of the appended claims.

1. A Wnt culture system comprising:
minimal serum culture medium;
a biologically active Wnt polypeptide secreted into the minimal serum culture medium; and
cells from an engineered cell line transfected with an expression vector encoding the biologically active Wnt polypeptide, wherein the cells are grown in the presence of the minimal serum culture medium.

2-35. (canceled)

36. A method of preparing a liposomal Wnt polypeptide, comprising:
(a) incubating an isolated Wnt polypeptide with a plurality of chaperones to generate a Wnt polypeptide-chaperone complex;
(b) separating the Wnt polypeptide-chaperone complex from non-complexed chaperones; and
(c) contacting the Wnt polypeptide-chaperone complex with an aqueous solution of liposomes to generate the liposomal Wnt polypeptide.

37-89. (canceled)

90. An in vitro method of producing a biologically active Wnt polypeptide under a minimal serum condition, comprising:
culturing cells from an engineered cell line transfected with an expression vector encoding a Wnt polypeptide under the minimal serum condition; and
collecting secreted Wnt polypeptide from the culture medium under the minimal serum condition.

91-129. (canceled)


SEQUENCE LISTING (6 sequences)

SEQ ID NO: 1 - protein, 385 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCKNPGSRAGNSAHQPPHPQPPVRFHPPLRRAGKVP

SEQ ID NO: 2 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 3 - DNA, 2826 bp, Homo sapiens
atggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagctagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcggagcgcctccttaggagtggggttttatggtggatgaggcttcttcctggatggggcagagcttctcctgaccagggcaaggccccttccacgggggctgtggctctgggtgggcgtggcctgcataggctccttcctgtgggtggggcttctctgggaccaggctccaatggggcggggcttctctccgcgggtgggactcttccctgggaaccgccctcctgattaaggcgtggcttctgcaggaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaagggcctcccagcccccaaccccaagaccaagcttagtcctgggagaggacagggacttcgcagaggcaagcgaccgaggccctcccaaagaggcccgccctgcccgggctcccacaccgtcaggtactcctgccagggaactggcctgctgcgccccaggccccgcccgtctctgctctgctcagctgcgcccccttctttgcagctgcccagcccctcctccctgccctcgggtctccccacctgcactccatccagctacaggagagatagaagcctctcgtcccgtccctccctttcctccgcctgtccacagccccttaagggaaaggtaggaagagaggtccagccccccaggctgcccagagctgctggtctcatttgggggcgttcgggaggtttggggggcatcaaccccccgactgtgctgctcgcgaaggtcccacagccctgagatgggccggcccccttcctggcccctcatggcgggactggagaaatggtccgctttcctggagccaatggcccggcccctcctgactcatccgcctggcccgggaatgaatggggaggccgctgaacccacccggcccatatccctggttgcctcatggccagcgcccctcagcctctgccactgtgaaccggctcccaccctcaaggtgcggggagaagaagcggccaggcggggcgccccaagagcccaaaagagggcacaccgccatcctctgcctcaaattctgcgtttttggttttaatgttatatc

SEQ ID NO: 4 - protein, 694 aa, Homo sapiens
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPGEQPPSGSGHGRPPGARPPHRGGGRGGGGGDAAAPPARGGGGGGKARPPGGGAAPCEPGCQCRAPMVSVSSERHPLYNRVKTGQIANCALPCHNPFFSQDERAFTVFWIGLWSVLCFVSTFATVSTFLIDMERFKYPERPIIFLSACYLFVSVGYLVRLVAGHEKVACSGGAPGAGGAGGAGGAAAGAGAAGAGAGGPGGRGEYEELGAVEQHVRYETTGPALCTVVFLLVYFFGMASSIWWVILSLTWFLAAGMKWGNEAIAGYSQYFHLAAWLVPSVKSIAVLALSSVDGDPVAGICYVGNQSLDNLRGFVLAPLVIYLFIGTMFLLAGFVSLFRIRSVIKQQDGPTKTHKLEKLMIRLGLFTVLYTVPAAVVVACLFYEQHNRPRWEATHNCPCLRDLQPDQARRPDYAVFMLKYFMCLVVGITSGVWVWSGKTLESWRSLCTRCCWASKGAAVGGGAGATAAGGGGGPGGGGGGGPGGGGGPGGGGGSLYSDVSTGLTWRSGTASSVSYPKQMPLSQV

SEQ ID NO: 5 - protein, 385 aa, Artificial Sequence
  Synthetic polypeptide
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 6 aa, Artificial Sequence
  Synthetic polypeptide
IEGRMD